(19) United States
(12) Patent Application Publication
(10) Pub. No.: US 2021/0280078 A1
(43) Pub. Date: Sep. 9, 2021

(54) METHOD AND SYSTEM FOR REMOTELY COACHING A SET OF PARTICIPANTS

(71) Applicant: Omada Health, Inc., San Francisco, CA (US)

(72) Inventors: Ryan Quan, San Francisco, CA (US); Devin Ellsworth, San Francisco, CA (US); Adrian James, San Francisco, CA (US); Mike Tadlock, San Francisco, CA (US); Stephen Hess, San Francisco, CA (US); Tina Yeung, San Francisco, CA (US); Luke Armistead, San Francisco, CA (US); Jonathan Wrobel, San Francisco, CA (US)

(21) Appl. No.: 17/195,156

(22) Filed: Mar. 8, 2021

Related U.S. Application Data
(60) Provisional application No. 62/986,385, filed on Mar. 6, 2020.

(51) Int. Cl. G09B 19/00; G16H 40/20; G16H 50/20; G16H 50/70; G16H 10/60; G16H 40/67; G06F 16/245

(52) U.S. Cl. CPC G09B 19/00; G16H 40/20; G16H 50/20; G06F 40/35; G16H 10/60; G16H 40/67; G06F 16/245; G16H 50/70

(57) ABSTRACT

A system for coaching a set of participants includes a coach-participant interface including a coach dashboard and optionally a participant dashboard. A method for coaching a set of participants includes: collecting a set of inputs associated with a set of one or more participants; for each of the set of participants, determining a set of one or more scores associated with the participant based on the set of inputs; and organizing the set of participants based on the scores. Additionally, the method can include any or all of: assigning a set of tags and/or labels and/or any other suitable process(es).

### CROSS-REFERENCE TO RELATED APPLICATIONS

This application claims the benefit of U.S. Provisional Application No. 62/986,385, filed 6 Mar. 2020, which is incorporated in its entirety by this reference.

### TECHNICAL FIELD

This invention relates generally to the health field, and more specifically to a new and useful system and method for assisting a coach in remotely coaching a set of participants in the health field.

### BACKGROUND

In current remote coaching platforms with human coaches, the scalability is mainly limited by the number of participants to which a coach (equivalently referred to herein as an agent) has the bandwidth to provide adequate attention. While some platforms have switched to fully automatically-generated coach communications, the personal touch and adaptability of a human coach is lost.

Thus, there is a need in the remote coaching field to preserve the personal touch of a human coach while helping him or her efficiently manage and thereby scale the number of participants that he or she can coach.

### BRIEF DESCRIPTION OF THE FIGURES

FIG. 1 is a schematic of a system for remotely coaching a set of participants.

FIG. 2 is a schematic of a method for remotely coaching a set of participants.

FIG. 3 is a schematic of a variation of a system for remotely coaching a set of participants.

FIG. 4 depicts a variation of a coach dashboard.

FIG. 5 depicts a variation of a message board of a coach dashboard.

FIG. 6 depicts a variation of a participant profile.

FIGS. 7 and 8 depict variations of a set of surveys provided to a participant.

FIG. 9 depicts a variation of a meals and activity tracker including a set of inputs received from a participant.

FIG. 10 depicts a variation of a set of tools provided at a coach dashboard, which can assist a coach in efficiently communicating with participants.

FIGS. 11-13 depict a variation of a coach dashboard.

FIG. 14 depicts a variation of information flow in the system and/or method.

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. Overview

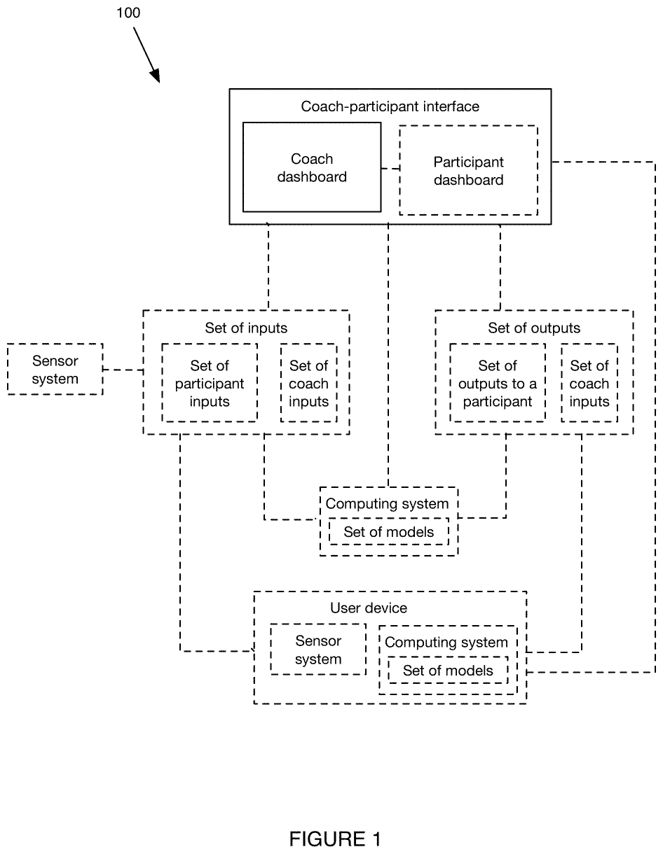

As shown in FIG. 1, a system 100 for remotely coaching a set of participants includes a coach-participant interface including a coach dashboard and optionally a participant dashboard. Additionally or alternatively, the system 100 can include and/or interface with any or all of: a set of inputs (e.g., from a participant, from a coach, etc.), a set of outputs (e.g., to a participant, to a coach, etc.), a set of models (e.g., to convert the set of inputs into the set of outputs), a computing system, a user device (e.g., to receive the set of inputs, to display the set of outputs, to display the dashboard, etc.), a sensor system (e.g., weight scale, step counter, accelerometer, etc.) to collect a set of one or more inputs from a set of participants, and/or any other suitable components.

Further additionally or alternatively, the system 100 can include any or all of the systems, components, embodiments, and examples described in U.S. application Ser. No. 15/667,218, filed 2 Aug. 2017, which is incorporated herein in its entirety by this reference.

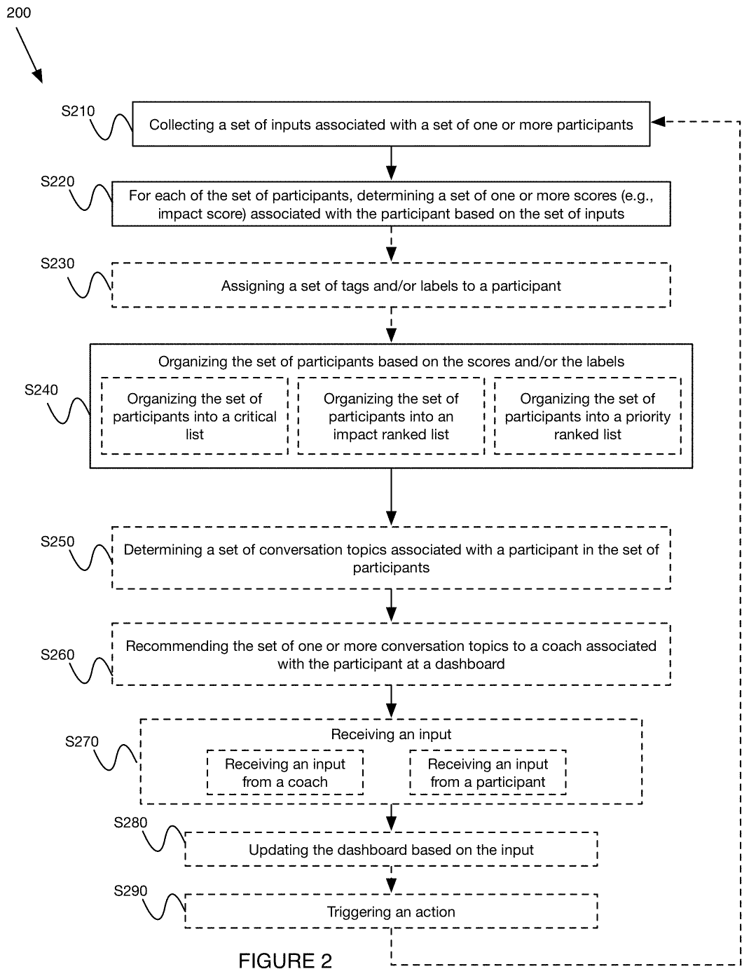

As shown in FIG. 2, a method 200 for remotely coaching a set of participants includes: collecting a set of inputs associated with a set of one or more participants S210; for each of the set of participants, determining a set of one or more scores (e.g., impact score) associated with the participant based on the set of inputs S220; and organizing the set of participants based on the scores S240. Additionally, the method 200 can include any or all of: assigning a set of tags and/or labels to any or all of the set of participants (e.g., based on the set of scores) S230; determining a set of conversation topics associated with a participant of the set of participants S250; recommending the set of one or more conversation topics to a coach associated with the participant at a dashboard S260; receiving an input S270; updating the dashboard based on the input S280; triggering an action S290; and/or any other suitable process(es).

Further additionally or alternatively, the method 200 can include any or all of the methods, processes, embodiments, and examples described in U.S. application Ser. No. 15/667,218, filed 2 Aug. 2017, which is incorporated herein in its entirety by this reference.

The method 200 can be performed with a system as described above and/or any other suitable system.

### 2. Benefits

The system and method for remotely coaching a set of participants can confer several benefits over current systems and methods.

In a first set of variations, the system and/or method confers the benefit of enabling a set of coaches to efficiently provide information to users with a personal touch that is correct and optimal for the participant (equivalently referred to herein as a user or a patient). In a set of specific examples, for instance, the method includes automatically generating a set of discussion topics for each participant to guide a coach in effectively and efficiently interacting with each participant.

In a second set of variations, additional or alternative to the first, the system and/or method confers the benefit of predicting, preventing, and/or reducing churn (e.g., participant quitting, disinterest, diminishing or otherwise changing engagement, etc.) of participants. The inventors have discovered, for instance, that a common preemptive sign that a participant is going to leave a program is a lapse in performing deliverables for the program, such as any or all of: tracking meals, doing weigh-ins, and/or any other deliverables. The system and/or method can enable detection of the onset of a preemptive sign and recommend communications to coaches that re-engages the participant prior to losing them. In specific examples, for instance, upon detecting a likelihood above a threshold that a participant will disengage from the program, one or more coaches can be notified and encouraged to take measures (e.g., increasing communication, reminding the participant to record meals, etc.) to reduce the likelihood that the participant will disengage. Additionally or alternatively, one or more automated actions can be taken, such as an automated message sent to the participant.

In a third set of variations, additional or alternative to those described above, the system and/or method confers the benefit of recommending communications to coaches to send to a participant based on a set of outcome key drivers. In specific examples, for instance, communications are recommended to coaches which promote weight loss by the set of participants.

In a fourth set of variations, additional or alternative to those described above, the system and/or method confers the benefit of increasing an efficiency of a set of coaches through any or all of: directing a coach to the highest impact participants (e.g., as determined through a set of models and/or algorithms); recommending topics and/or specific messages to a coach that are predicted to be effective with the participant; automating core workflows (e.g., auto-populating messages, auto-populating messages that are not repetitive with respect to previous messages, etc.); and/or performing any other suitable functions. In a set of specific examples, for instance, the dashboard enables a coach to know who to reach out to, what to say, and what information is relevant (e.g., what participants and/or conversation topics are highest priority), which provides the coach with any or all of: a set of participant rankings (e.g., indicating which participant to reach out to first), a set of recommended conversation topics, an auto-generated or auto-filled set of responses for a coach to use in conversation, and/or any other suitable information.

In a fifth set of variations, additional or alternative to those described above, the system and/or method confers the benefit of assisting a coach in his or her actions with a participant while still maintaining a personal touch and/or a characteristic feature (e.g., tone, writing style, etc.) of the coach. In specific examples, the method enables auto-generated and/or auto-filled messages to be provided to a coach and/or to a participant associated with the coach, wherein the messages generated can be configured to: not be repetitive with respect to previous messages from the coach, preserve a writing/conversation style of a coach, and/or perform any other suitable function.

In a sixth set of variations, additional or alternative to those described above, the system and/or method confers the benefit of automatically organizing participants to prioritize the coach's engagement with the most engaged participants, as the inventors have discovered that these participants benefit the most from engagement with coaches. In specific examples, the method includes determining a rapport score for each participant based on features of his or her messaging with the coach.

In a seventh set of variations, additional or alternative to those described above, the system and/or method confers the benefit of providing a robust and scalable tool for coaches to help optimize his or her interactions with participants without requiring a large amount of training data. In specific examples, for instance, the system and/or method optimizes these interactions with a set of trained uplift models, wherein the uplift models require a relatively small amount of data for training (e.g., relative to reinforcement learning (RL) and/or other types of machine learning) and do not require to be manually programmed and updated as in rule-based approaches.

In an eighth set of variations, additional or alternative to those described above, the system and/or method confers the benefit of partially or fully automating the organization and prioritization of a set of participants to a coach at a coach interface (e.g., coach dashboard). In some examples, for instance, the system and/or method automatically organizes participants into a set of lists which can be directly provided, determined and provided in response to a search term from the coach, otherwise provided, and/or any combination.

In a ninth set of variations, additional or alternative to those described above, the system and/or method confers the benefit of prioritizing the participants who show the greatest interest in a digital health program, which the inventors have discovered leads to the most optimal overall outcomes. In specific examples, the system and/or method automatically determines a rapport between a coach and each of his or her assigned participants, wherein the system and/or method are configured to prioritize coach interactions with those participants having the highest rapport.

Additionally or alternatively, the system and method can confer any other benefits.

### 3. System

As shown in FIG. 1, the system 100 includes a system 100 for remotely coaching a set of participants includes a coach-participant interface including a coach dashboard and optionally a participant dashboard. Additionally, the system 100 can include and/or be configured to interface with any or all of: a set of inputs, a set of outputs, a sensor system, a computing system and/or a set of models, a user device, and/or any other suitable components. Further additionally or alternatively, the system 100 can include any or all of the systems, components, embodiments, and examples described in U.S. application Ser. No. 15/667,218, filed 2 Aug. 2017, which is incorporated herein in its entirety by this reference.

### 3.1 System—Components

The system 100 includes a coach-participant interface, which functions to provide a platform with which a coach can interact with a set of participants. Additionally or alternatively, the coach-participant interface can include a participant dashboard, which functions to provide a platform with which a participant can interaction with his or her coach. Further additionally or alternatively, the coach-participant interface can perform any or all of the following functions: providing information to a set of one or more coaches (e.g., informing them of the status of a set of users); providing information to a set of one or more participants (e.g., providing them with information associated with their health regimen(s), providing them with information from their coaches, providing them with resources, etc.); collecting information from a set of participants (e.g., aggregated information with which to update and/or train a set of models, etc.); collecting information from a set of coaches (e.g., with which to train a set of coaches); improve an efficiency of a set of coaches (e.g., through prioritization of coach tasks, through the auto-generation of a set of recommended topics, through easy-to-interpret visualizations, etc.); and/or perform any other suitable function(s).

A coach refers to an agent using the system to engage with and coach a set of one or more participants in achieving one or more goals of the participant. The goals can be any or all of: selected by the participant, recommended by a coach, recommended and/or required by a third party (e.g., medical professional, employer, family member, etc.), automatically determined (e.g., by one or trained models), predicted, and/or otherwise determined.

The coach can refer to either or both of a human agent and a non-human agent (e.g., automated agent, robot, chat bot, etc.). For human coaches, the coach can be any or all of: a healthcare professional, a fitness trainer, a nutritionist, a therapist, a medical professional (e.g., doctor, physician, etc.), and/or any other individual affiliated or not affiliated with a health and/or fitness profession. In preferred variations, a participant is assigned to a human agent which remotely (and/or locally) coaches them. Additionally or alternatively, the human agent can be assisted by a non-human agent and/or a set of automated processes, which can assist or replace the human agent in performing certain tasks, such as any or all of: replying to a participant with an auto-generated message (e.g., in absence of the human agent, upon request of the human agent, in an urgent scenario, etc.), auto-filling a message initiated by a human agent, and/or performing any other suitable actions.

In a set of specific examples, specific messages can be recommended to coaches, wherein the specific messages are recommended based on outcomes predictions (e.g., based on a set of trained models). A set of chatbots can be optimized based on learnings from these messages and/or feedback from human coaches.

The dashboard or dashboards of the coach-participant interface are preferably implemented on one or more user devices (e.g., associated with the coach, associated with the participant, etc.), such as through a client application executing on a user device. Additionally or alternatively, the dashboards can be otherwise implemented.

The coach-participant interface includes a coach dashboard (equivalently referred to herein as a coach interface), which functions to provide information associated with a set of one or more participants to the coach or coaches associated with the set of participants. The dashboard preferably additionally functions to provide a set of tools for the coach (e.g., recommended conversation topics, stored conversation topics, etc.) and to receive inputs from the coach, but can additionally or alternatively perform any other functions.

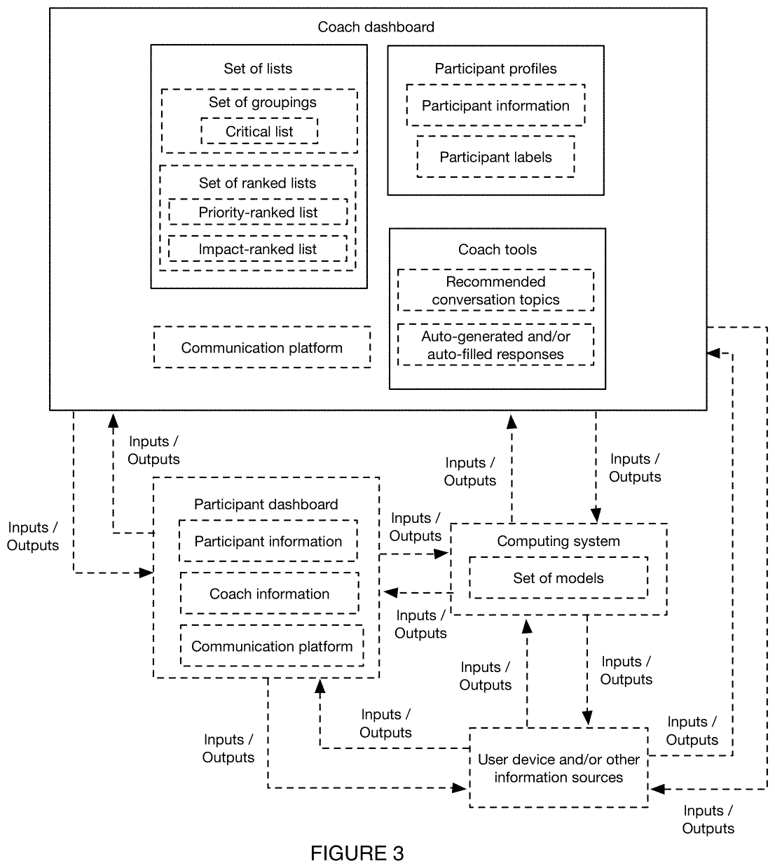

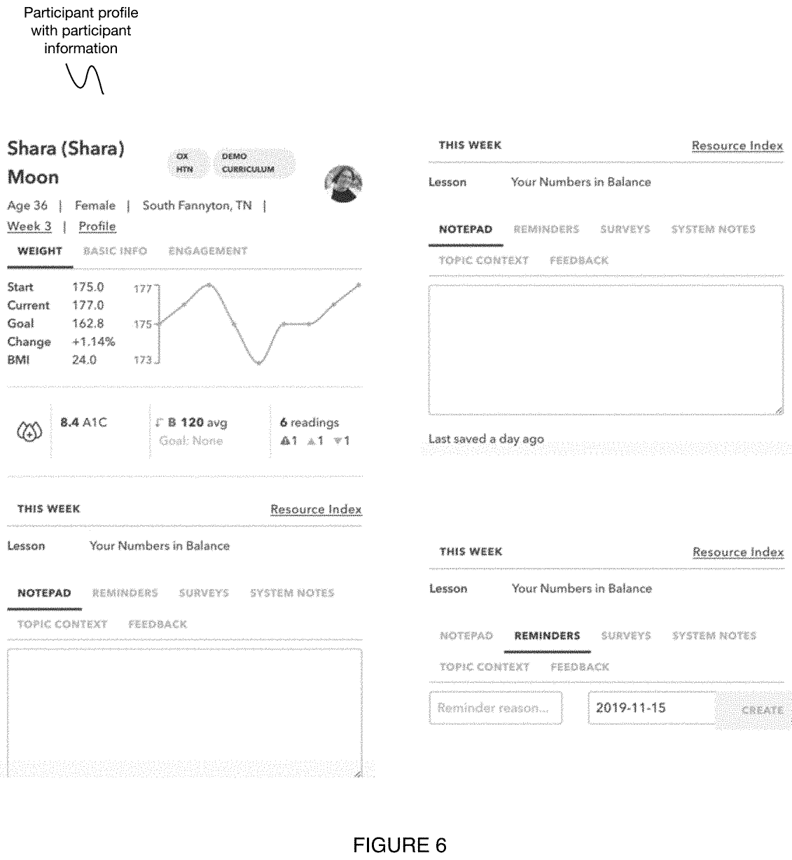

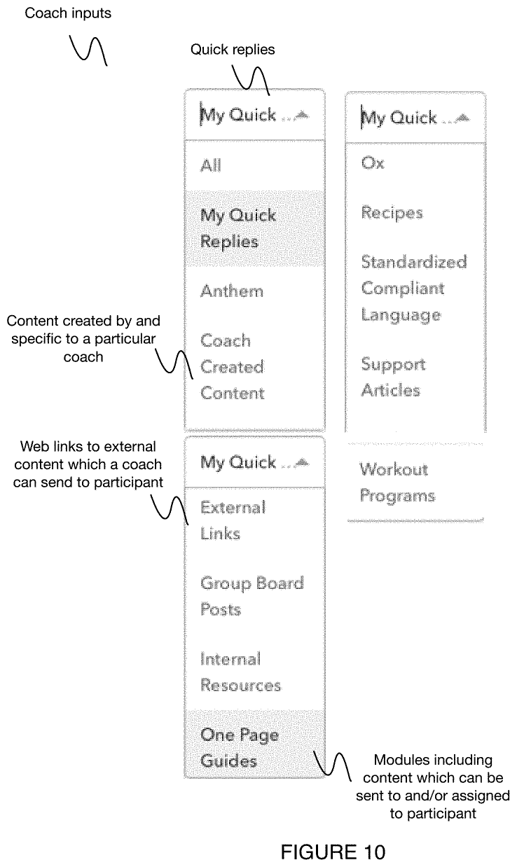

In preferred variations (e.g., as shown in FIG. 3), the coach dashboard includes any or all of: a set of lists (e.g., as described below) configured to direct the coach's attention to the set of participants a coach should reach out to and optionally the order in which a coach should reach out to the participants; access to participant profiles (e.g., as shown in FIG. 6) associated with each of a set of participants, which can function to provide the coach with numerous types of information associated with each participant and/or an aggregated set of participants (e.g., all participants assigned to the coach, all participants in the program, etc.); a set of coach tools (e.g., a set of recommended conversation topics, a set of auto-generated and/or auto-filled responses, as shown in FIG. 10, etc.); and a communication platform configured to enable communication between the coach and his or her participants. Additionally or alternatively, the coach dashboard can include any other suitable features and/or information.

The participant information associated with each participant (e.g., and contained in the participant profile on the coach dashboard, as contained in a participant dashboard, as received at a remote computing system, etc.) can include individual and/or aggregated information, any of which can include any or all of: demographic information (e.g., race, gender, age, socioeconomic information, etc.), participant identifiers (e.g., name, photo, etc.), medical and/or clinical information (e.g., test results, physician notes, pathologies, etc.), biometric information (e.g., heart rate, blood pressure, blood glucose level, hemoglobin level, etc.), weight, meals consumed by the participant, fitness activities performed by the participant, participant goals (e.g., run a marathon, lose ten pounds, etc.), a set of tags and/or labels (e.g., as described below) associated with the patient, a set of scores associated with the patient (e.g., as described below), and/or any other suitable information.

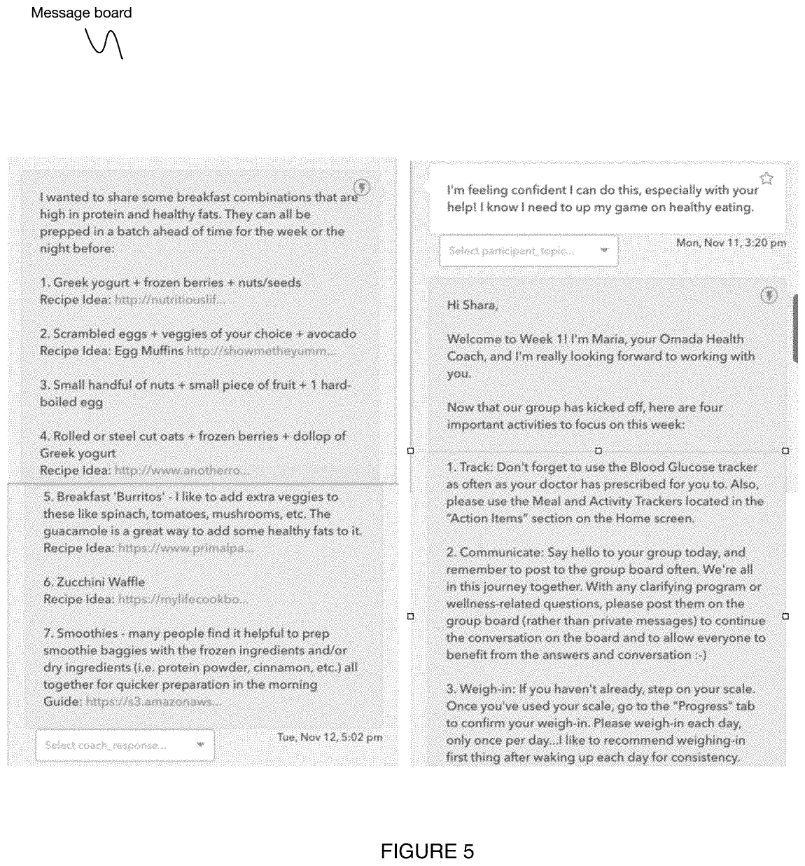

The communication platform preferably includes a messaging platform (equivalently referred to herein as a message platform) (e.g., as shown in FIG. 5) through which coaches and participants can exchange messages. Additionally or alternatively, the communication platform can be configured to enable calls (e.g., telephone calls, video calls, etc.) between coaches and participants, messages and/or calls between coaches and medical professionals, messages and/or calls between participants and medical professionals (e.g., as initiated by a coach), messages and/or calls between coaches (e.g., for coaches to collaborate, share advice, cover for each other, etc.), and/or any other suitable communication. The communication platform is preferably implemented through one or more dashboards (e.g., coach dashboard, participant dashboard, etc.), but can additionally or alternatively be implemented through a user device (e.g., associated with the participant, associated with the coach, implementing the dashboard, etc.) and/or any client application executing on the user device(s) and/or any other suitable device.

The system 100 can optionally receive any number of inputs, which can be received from any or all of: a set of one or more participants, a set of one or more coaches, a computing system (e.g., as generated by the set of models), external information sources (e.g., census information, online information, etc.), a user device and/or client applications (e.g., a meal tracker client application, a fitness tracker client application, sleep tracker client application, etc.), supplementary devices (e.g., smart watch, blood pressure monitor, glucose meter, insulin pump sensor, scale for measuring user weight, etc.), databases (e.g., lookup tables, medical files, etc.), and/or any other suitable information sources.

The set of inputs can be received at any or all of: one or more user devices, a computing system (e.g., a remote computing system, computing system of a user device, etc.) and/or server (e.g., remote server, cloud-based server, etc.), a dashboard (e.g., coach dashboard, participant dashboard, etc.), and/or any other suitable locations.

Any or all of the set of inputs can be collected continuously (e.g., at a predetermined frequency, upon being input into a dashboard by a coach and/or a participant, at an intermittent frequency, etc.). Additionally or alternatively, one or more inputs can be collected once (e.g., participant demographic information), in response to a trigger, upon being updated, and/or at any other suitable times.

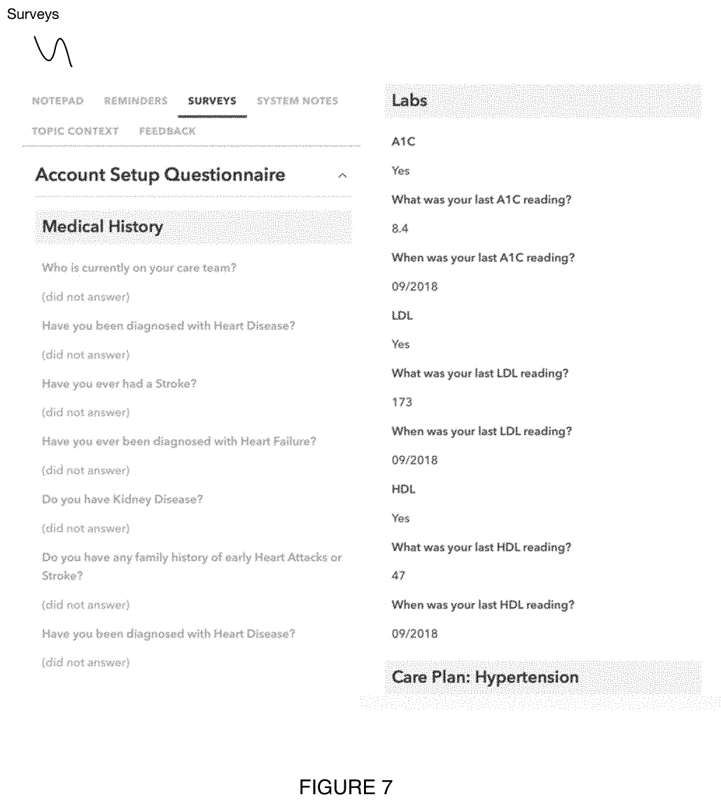

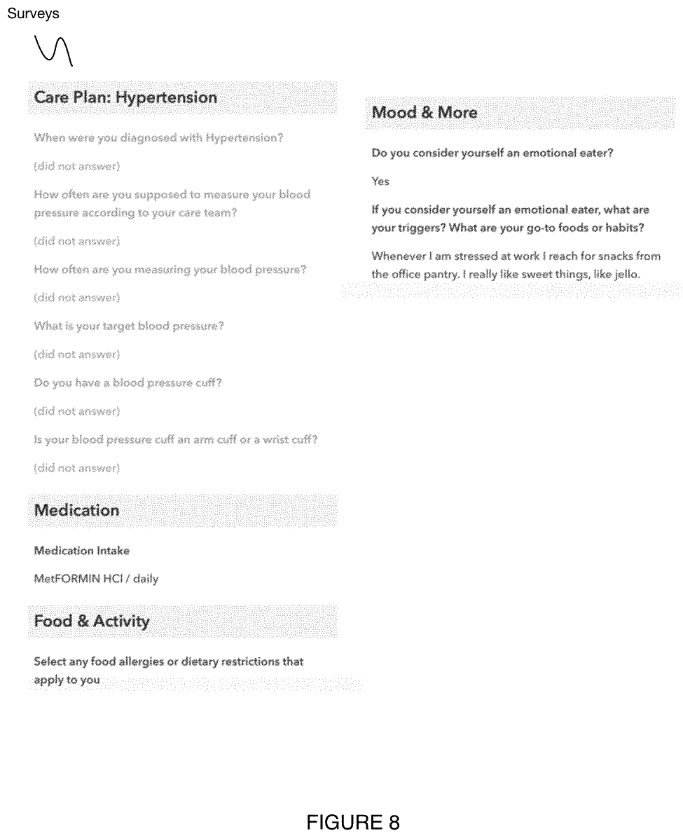

The set of inputs can include any number of coach inputs, which are received from one or more coaches and/or otherwise associated with one or more coaches (e.g., automatically generated for the coach) and can function to guide the participants in their regimens and achieving their health goals or other goals. Coach inputs can include any or all of: articles to send to a participant (e.g., to help them with a particular topic, to help them lose weight, to guide them in performing a fitness routine, etc.), surveys (e.g., as shown in FIGS. 7 and 8) to send to a participant, messages to send to a participant, reminders to send to a participant, and/or any other suitable inputs. Additionally or alternatively, the coach inputs can include notes and/or other documentation configured to help a coach remember and/or track information, such as the coach's thoughts about a participant (e.g., his or her progress, topics to check in on, etc.). Further additionally or alternatively, the set of coach inputs can include coach preferences (e.g., dashboard format preferences) and/or coach information (e.g., demographic information, historical information, prior messages, etc.).

The coach inputs can be any or all of: determined (e.g., selected, developed, etc.) by the coach, determined by a computing system (e.g., through a set of models), and/or any combination of both (e.g., suggested to a coach and selected by the coach).

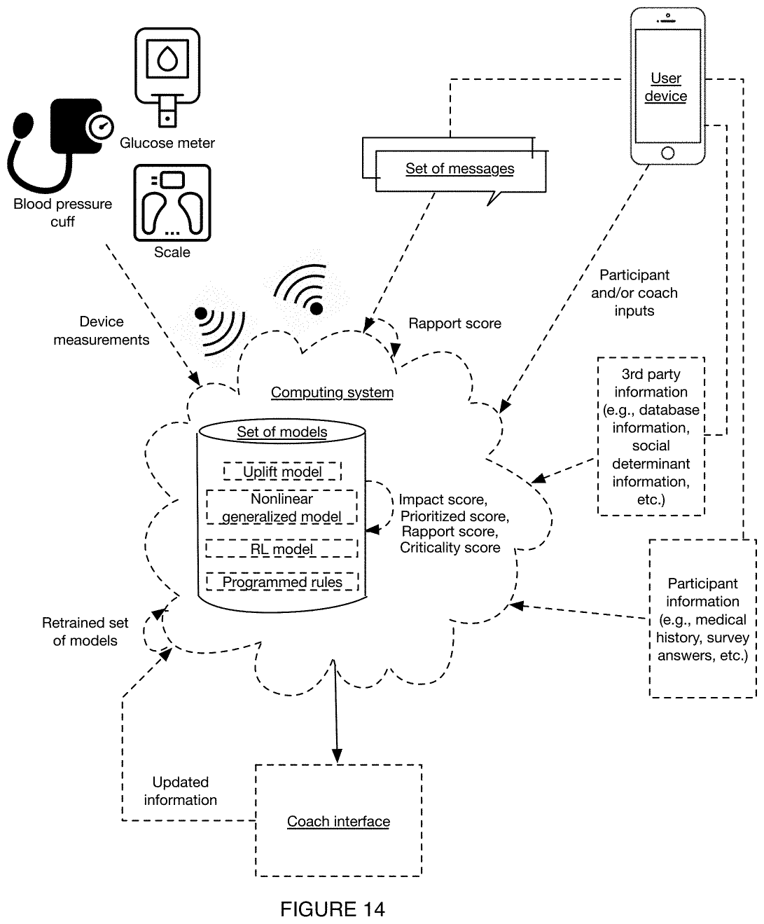

The set of inputs can include any number of participant inputs (e.g., as shown in FIG. 14), which are received from participants and/or otherwise associated with the participants (e.g., received from devices of the participant, received from 3^{rd} party information sources, etc.) and can function to provide information to a coach, provide information for a set of models to analyze, provide information to train a set of models, provide information to be aggregated with information from other users, and/or perform any other suitable function.

The participant inputs can be any or all of: provided by the participant, provided (e.g., automatically provided, manually provided, etc.) by an external source (e.g., healthcare facility visited by the participant, sampled at a sensor system of a user device and/or external device, 3^{rd} party system and/or database, etc.), determined by a computing system (e.g., through a set of models), and/or any combination (e.g., provided by a participant and processed at a computing system).

The participant inputs can optionally include one or more clinical outcomes (e.g., diabetes, depression risk, medical records, etc.). In some variations, clinical outcomes are received in response to a user providing consent for the system to access clinical records (e.g., from a healthcare facility, from a medical records database, etc.). Additionally or alternatively, the clinical outcomes can be otherwise received (e.g., directly from the participant, etc.).

The participant inputs can optionally include participant preferences, such as goals and motivations (e.g., look better, lose weight, complete a particular fitness activity, increase strength, increase endurance, reduce severity of a medical condition, etc.). The participant preferences can be determined based on any or all of: clickstream data (e.g., within a client application containing the participant dashboard, within external client applications, on the internet, etc.), survey information, and/or any other suitable sources.

The participant inputs can optionally include social determinants, which can function to tailor the coaching provided to the participant. These can include any or all of: regional parameters associated with the participant's location (e.g., food desert, food oasis, proximity to a healthy food source), lifestyle parameters (e.g., commute time, occupation, etc.), and/or any other suitable parameters. The social determinants can be collected from the participant, from 3^{rd} party and/or public data sources (e.g., census information, neighborhood information, employment information, LinkedIn, internet searches, etc.), and/or from any other information sources. In some variations, for instance, any or all of these social determinants can significantly affect a participant's ability or predicted ability to make positive change in leading a health lifestyle. If the participant lives in a food desert, for instance, the coach may provide resources specific to this type of region, such as recipes which do not include ingredients difficult for the participant to easily access.

The participant inputs can optionally include time-varying behaviors (e.g., habits), such as temporal seasonality (e.g., weekly seasonality where participant is offline for a week with no access to scale for weigh-ins due to an occupation such as a flight attendant, truck driver, etc.; monthly seasonality; etc.). The time-varying behaviors can be determined based on direct inputs from the participant, information collected at client applications (e.g., calendar), employment information of the participant, sensor information associated with the participant (e.g., elevation, GPS information, pedometer information, etc.), and/or any other suitable information.

The participant inputs can include lifestyle data, such as any or all of: food consumed by the participant (e.g., tracked meals, tracked calories, photos of food, etc.), exercise performed by the participant (e.g., as input by the participant, as detected through one or more sensors such as a pedometer, etc.), and/or any other suitable information.

In some variations, for instance, the system 100 can receive either or both of manually-generated and passive inputs collected from a participant and/or any number of devices (e.g., user devices, medical devices, sensors, etc.) associated with the participant, wherein the manually-generated inputs can include messages, manually-entered information (e.g., demographic information, survey answers, meal tracking information, etc.), user selections (e.g., at the participant dashboard), and/or any other information, and wherein the passive inputs can include automatically-collected and/or automatically generated information (e.g., from one or more sensors of supplementary devices such as biometric information, information from one or more client applications or databases such as user's location, activity information such as login activity and/or engagement activity at the user's dashboard, etc.). Additionally or alternatively, the system 100 can receive any other suitable information.

In specific examples, participants in the program can optionally utilize any number of integrated devices to collect inputs (e.g., in additional to inputs collected at client applications and/or user interfaces), wherein the particular devices can be determined, for instance, based on any or all of: health conditions of the participant (e.g., glucose meter for diabetic participants), goals of the participant (e.g., meal tracker application for participants with a goal of weight loss, connected smart scale for participants with a goal of weight management, etc.), financial resources of the participant, and/or any other participant and/or coach preferences. Additionally or alternatively, devices can be any or all of: predetermined for the participants, the same for all participants, randomly used, and/or otherwise used.

The system 100 can optionally produce any number of outputs, such as any or all of the tools provided to the coaches (e.g., as described above), information provided to the participants (e.g., surveys, recommendations, messages from a coach, etc.), information provided to the coaches, and/or any other suitable outputs. The outputs can be produced by a computing system (e.g., by a set of models), received from a coach, received from a participant, received from an external data source, and/or otherwise produced.

The system 100 can include and/or interface with any number of user devices, which can individually and/or collectively function to: support a dashboard, support a client application (e.g., including the dashboard), receive any or all of a set of inputs (e.g., from a set of client applications), produce any or all of a set of outputs, and/or perform any other suitable functions. Examples of the user device include a tablet, smartphone, mobile phone, laptop, watch, wearable device (e.g., glasses), or any other suitable user device. The user device can include power storage (e.g., a battery), processing systems (e.g., CPU, GPU, memory, etc.), user outputs (e.g., display, speaker, vibration mechanism, etc.), user inputs (e.g., a keyboard, touchscreen, microphone, etc.), a location system (e.g., a GPS system), sensors (e.g., optical sensors, such as light sensors and cameras, orientation sensors, such as accelerometers, gyroscopes, and altimeters, audio sensors, such as microphones, magnetometers, etc.), data communication system (e.g., a WiFi transceiver(s), Bluetooth transceiver(s), cellular transceiver(s), etc.), or any other suitable component.

The system 100 can include and/or interface with any number of sensor systems. The sensor systems can be part of a user device (e.g., accelerometer, location sensor, camera, microphone, etc.), part of a supplemental device (e.g., scale, pedometer, step counter, etc.), and/or part of any other suitable components. In some variations, for instance, one or more participant inputs are received from the sensor system. In specific examples, the sensor system includes any or all of: a connected scale for monitoring of a user's weight, a fitness tracker (e.g., step counter), a sleep tracker (e.g., as part of a smart watch), a medical device (e.g., glucose monitor, blood pressure cuff, etc.), and/or any other suitable sensor systems. Additionally or alternatively, any or all of the sensor systems and/or supplementary devices described in U.S. application Ser. No. 15/667,218, filed 2 Aug. 2017, which is incorporated herein in its entirety by this reference, can be included.

The system 100 can include or interface with any number of computing systems, such as any or all of: a computer, a processor, an analysis engine, a server, and/or any other suitable system(s). The computing system can be any or all of: remote (e.g., cloud computing system), local (e.g., onboard a user device, distributed among multiple user devices, etc.), or any combination of both.

The computing system preferably implements a set of one or more models, which function to assist a coach in communicating with his or her set of participants. The set of models can include a set of trained models (e.g., deep learning models, machine learning models, neural networks, etc.), which are preferably trained on and/or optimized based on any or all of: desired participant behaviors, desired participant health/fitness outcomes (e.g., participant-specific health goals, participant-agnostic health goals, health goal selected by the participant, health goal selected by a coach or healthcare professional, etc.), participant goals and/or coach goals for the participant, and/or any other suitable information. Additionally or alternatively, the set of models can include untrained models, and/or any combination of both. Further additionally or alternatively, the set of models can be implemented by any other components or combination of components.

The set of models are preferably configured to increase an efficiency of the coaches, such as through determining and/or prioritizing which participants to reach out to (e.g., as described in the critical list described below). In specific examples, for instance, when revenue is tied to health outcomes but coaches each have a finite amount of time, knowing who needs to be contacted and/or who will benefit the most from being contacted can help coaches use their time most efficiently, as well as confer other benefits such as ensuring that the participants who need and/or want to be reached out to are contacted by the coach. The set of models can optionally additionally or alternatively configured to determine a set of recommended discussion topics (e.g., as described below). Additionally or alternatively, the set of models can determine in which order a set of participants should be reached out to by a coach (e.g., as described in the set of ranked lists below), how often a participant should be messaged, what type of interaction a coach should have with a participant (e.g., sending a message vs. an information packet), what interactions will most likely lead to a particular behavior change, how to batch participants (e.g., based on the language used in participant conversations), what to say in a message (e.g., through partial and/or full automation), and/or any other suitable information.

The set of models preferably include one or more trained (e.g., supervised, unsupervised, semi-supervised, etc.) machine learning models (e.g., deep learning models, etc.), but can additionally or alternatively include and/or implement any or all of: untrained models, rule-based models (e.g., programmed models and/or algorithms and/or equations), rule sets, criteria, thresholds, and/or any other suitable tools.

The set of models preferably implements predictive modeling, which functions to predict the incremental impact of a coach input and/or interaction on the participant's behavior. This can be implemented, for instance, to determine scenarios in which, if the coach is there and interacting with the participant, the participant would do better (e.g., move closer to achieving goals, exhibit better engagement with coach, etc.). In preferred variations, the predictive modeling technique includes uplift modeling (equivalently referred to herein as incremental building, true lift modeling, net modeling, etc.). Additionally or alternatively, any other predictive modeling or other type of modeling can be used.

The set of predictive models can receive (and/or be trained based on) any or all of the inputs described above. In some variations, the set of predictive models receives, for each participant, any or all of the following information: historical information associated with the participant (e.g., coach's previous interactions with user, correlations between coach's previous interactions with the participant and a participant progress level, scores and/or progress levels associated with the participant, etc.), demographic information, and/or any other suitable participant information. The set of predictive models can additionally or alternatively receive aggregated information from a set of multiple participants, which can be used to predict the response of a participant based on historical responses of other participants (e.g., with shared attributes, which similar demographic information, with a similar engagement level with coach, etc.). Further additionally or alternatively, the set of predictive models can include any other suitable information.

In preferred variations, an output of the predictive models is one or more impact-ranked lists (e.g., as described below), which function to rank participants based on a predicted impact that an interaction with and/or input from a coach will have on the participant. In specific examples, participants associated with the greatest potential for positive change and/or potential for greatest positive change are ranked highest in the impact ranked list.

The set of models can additionally or alternatively implement regression modeling, which functions to predict multiple variables, such as multiple correlated dependent variables. In preferred variations, the set of regression models includes a set of multivariate linear regression models, with the variables including any number and type of metrics (markers) associated with the success of a participant. These can include an engagement level of a participant with a coach, which can be measured through any or all of: participant's usage of a client application, time spent interacting with the participant dashboard, number of messages exchanged with a coach, length of messages exchanged with a coach, time between interactions with a coach, frequency of interactions with a coach, and/or any other suitable parameters. In a first set of variations, for instance, one or more uplift models are used which implement one or more regression techniques (e.g., linear regression, non-linear regression, etc.). Additionally or alternatively, regression modeling can be used elsewhere, the uplift models and/or other predictive models can be otherwise implemented, and/or any other suitable models can be used.

The set of models can further additionally or alternatively include one or more topic models which implement a topic modeling approach (e.g., unsupervised topic modeling, supervised topic classification, Correlation Explanation [CorEx], etc.) to assess one or more features (e.g., tone, emotional content, bonding, rapport, etc.) of messages exchanged between the coach and a participant. This can then optionally be used, for instance, to determine which participants the coach as the best rapport with (e.g., best connection, best engagement, etc.), as the inventors have discovered that participants with the best rapport benefit the most from coaches reaching out to them. In specific examples, for instance, one or more scores (e.g., impact score) can take this into account to prioritize coaches reaching out to participants with whom they have the best rapport. Additionally or alternatively, participants can be otherwise prioritized and/or incentivized, and/or the topic modeling can be otherwise suitably implemented, such as to determine which topics the coach has covered in his or her conversations with the participant.

In specific examples, a CorEx model is used to score the topic and/or sentiment of messages from a coach to a participant based on the raw text of the messages.

The set of models can further additionally or alternatively include models with which to determine a likelihood that a participant will respond favorably to a notification (e.g., reminder) from a coach at a particular moment in time. This can be implemented, for instance, with one or more reinforcement learning models, which can determine a favorability likelihood score based on the participant's clickstream data (e.g., historical information showing his or her clickstream in response to a notification) and/or any other suitable information. Additionally or alternatively, any other suitable models can be used.

The set of models can further additionally or alternatively include models with which to determine one or more scores quantifying a predicted outcome associated with a participant, such as his or her progress (e.g., actual progress, predicted progress, etc.) in reaching a goal, a relative predicted change in one or more conditions (e.g., health conditions, diabetes, high blood pressure, etc.) or outcomes, and/or any other suitable metrics. The set of models for determining a predicted outcome score preferably include one or more linear models (e.g., generalized linear model), but can additionally or alternatively include one or more nonlinear models, other regression models, and/or any other suitable models. In specific examples, these models take into account participant demographic information, participant behavior information, and historical participant outcome information, but can additionally or alternatively take into account any other inputs.

The set of models can further additionally or alternatively include one or more models configured to assess food/meals associated with the participant, such as in variations in which a participant logs his or her meals in the digital health program. In some variations, for instance, the set of models includes a model which scores a healthiness and/or perceived healthiness associated with the participant's meal logs. This score can be determined with a linear model (e.g., generalized linear model) or any other suitable models (e.g., as described above). The healthiness score is preferably determined based on meal log information provided by the participant, but can additionally or alternatively be determined based on a self-reported healthiness score from the participant and/or any other metrics. The food/meal models can additionally or alternatively include one or more models configured to determine a food availability score, which functions to determine to what extend the participant lives within a food desert. This can be determined based on any suitable information associated with the participant, such as his or her home address, 3^{rd} party and/or open source data relating to food deserts, map information (e.g., shortest distance to health food store and/or grocery store with produce), and/or any other suitable information. In specific examples, for instance, the food availability score can be used in the method 200 to recommend particular recipes to a participant (e.g., based on which food types are readily available to the participant).

The set of models can further additionally or alternatively include any other suitable models, such as other machine learning models, programmed models (e.g., rule-based algorithms, rulesets, etc.), and/or any other algorithms, set of criteria, or tools.

Further additionally or alternatively, the set of models can include any other suitable models.

In a first variation of the set of models, the set of models includes one or more uplift models, wherein the set of models is configured to organize and/or prioritize participants (e.g., into a set of lists), which guide the coach to having the most impactful interactions with receptive participants.

Additionally or alternatively, the set of models can include any or all of: other predictive and/or regression models, topic models, reinforcement learning models, inverse reinforcement learning models, linear models (e.g., generalized linear models), nonlinear models, and/or any other suitable models.

The system 100 can additionally or alternatively include any other suitable components.

### 4. Method

As shown in FIG. 2, a method 200 for remotely coaching a set of participants includes: collecting a set of inputs associated with a set of one or more participants S210; for each of the set of participants, determining a set of one or more scores (e.g., impact score) associated with the participant based on the set of inputs S220; and organizing the set of participants based on the scores S240. Additionally, the method 200 can include any or all of: assigning a set of tags and/or labels to any or all of the set of participants (e.g., based on the set of scores) S230; determining a set of conversation topics associated with a participant of the set of participants S250; recommending the set of one or more conversation topics to a coach associated with the participant at a dashboard S260; receiving an input S270; updating the dashboard based on the input S280; triggering an action S290; and/or any other suitable process(es).

The participants are preferably coached with respect to one or more health and/or fitness regimens, which can function to help any or all of the participants in achieving a health and/or fitness goal, such as any or all of: a goal determined by the participant (e.g., a goal weight, a fitness activity to complete such as running a marathon, etc.); a goal determined by the coach (e.g., based on coach experience, based on the coach's interactions with the participant, based on the coach's interactions with an aggregated set of participants, etc.); a goal determined by a medical professional (e.g., lower cholesterol to a predetermined value, maintain a blood pressure below a predetermined value, achieve a BMI below a predetermined value, etc.), wherein the medical professional can be a coach or supplemental to a coach (e.g., the participant's primary care physician; a goal determined by a set of models and/or algorithms (e.g., deep learning models, uplift modeling, etc.); and/or any other suitable goals.

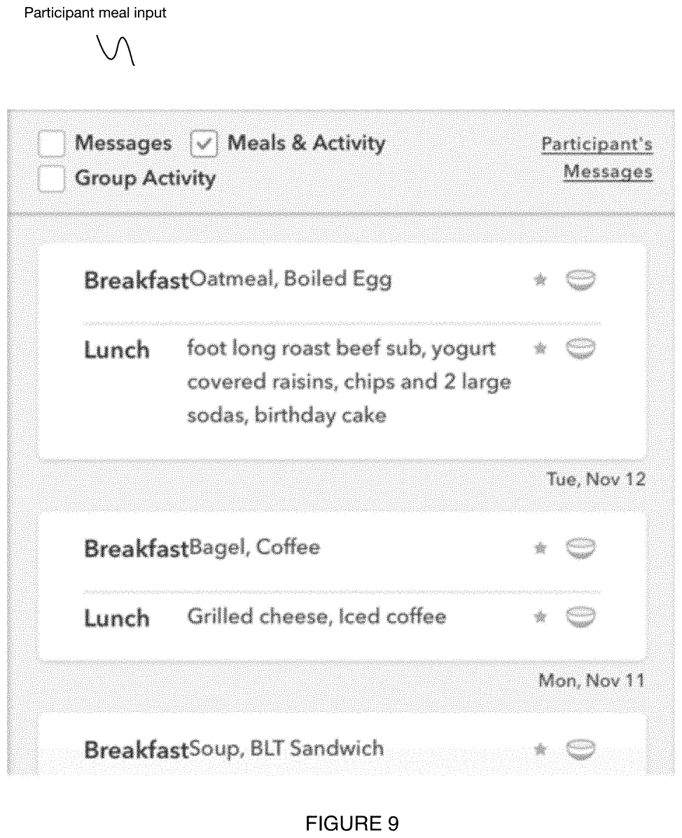

The health and/or fitness regimens can include any or all of: the collection and analysis (e.g., tracking) of information associated with the participants (e.g., meals/calories consumed by a participant as shown in FIG. 9, physical activities such as number of steps performed by a participant, the weight of a participant, the glucose level of a participant, the blood pressure of a participant, the heart rate of a participant, the blood sugar of a participant, a mood or emotion of a participant, a stress level of a participant, a sleep parameter of a participant, etc.); coach interactions with the participant (e.g., messages, notifications, reminders, conversations, etc.); coach suggestions and/or recommendations to a participant (e.g., recommended recipes, recommended fitness activity, etc.); input from a medical professional (e.g., coach, someone other than a coach, etc.); a health and/or fitness plan (e.g., dietary plan, fitness plan, etc.); and/or any other suitable features.

4.1 Method—Collecting a Set of Inputs Associated with a Set of One or More Participants S210

The method 200 can include collecting a set of inputs associated with a set of one or more participants S210, which functions to receive information with which to determine and/or just one or more features of the coach dashboard, such as, but not limited to, any or all of:

Inputs to the regimen can be collected from any or all of: one or more participants (e.g., input into a dashboard, input into a client application such as at a user device of the participant, etc.); one or more coaches; one or more medical professionals; a computing system (e.g., remote computing system, user device computing system, etc.); a database and/or storage (e.g., remote server, database including census information, etc.); and/or any other suitable sources. In some variations, 3^{rd} party information sources are utilized for data collection. In examples, for instance, pairing with a grocery store at which a participant shops can lead to the automatic collection and tracking of food being purchased by the user.

The inputs can include, but are not limited to, any or all of the inputs described above.

The set of inputs are preferably collected continuously throughout the method 200, further preferably throughout the entire engagement period of the participant with a coach, but can additionally or alternatively be collected intermittently, in response to a trigger, randomly, during an onboarding process, and/or at any other suitable times.

In preferred variations, the set of inputs includes at least a subset of the set of participant inputs described above, such as participant demographic information, social construct information, participant preferences, sensor information (e.g., weight, biometric information, glucose level, blood pressure value, etc.), lifestyle data (e.g., meal logging information, exercise information, etc.), program activity information (e.g., log-in activity, time spent participant in program, messaging frequency, messaging length, message tone, etc.), and/or participant historical information (e.g., previous interactions with coach). Additionally, the set of inputs can include coach inputs (e.g., coach messages to a participant) and/or any other suitable inputs. The set of inputs can be for a single participant, an aggregated set of participants, or any combination.

4.2 Method—for Each of the Set of Participants, Determining a Set of One or More Scores (e.g., Impact Score) Associated with the Participant Based on the Set of Inputs S220

The method 200 includes for each of the set of participants, determining a set of one or more scores (e.g., impact score) associated with the participant based on the set of inputs S220, which functions to assess a relative importance of reaching out to a particular participant, which in turn can function to help a coach rank or otherwise triage a set of participants and reach out to the group of participants in an optimally efficient way. Additionally or alternatively, any or all of the set of scores can be used for: determining a set of one or more lists for a coach to use when interacting with and/or prioritizing interactions with one or participants; organizing participants within one or more lists (e.g., in a ranked order); updating any or all of these lists; and/or performing any other functions.

S220 is preferably performed in response to S210, but can additionally or alternatively be performed at any other suitable times (e.g., prior to S210, multiple times, etc.). S220 can be performed at any or all of: continuously (e.g., at a predetermined frequency), at random intervals, in response to a trigger (e.g., receipt of new information associated with the participant, upon prompting by a coach or other individual, logging on of participant and/or coach associated with participant, etc.), at random intervals, and/or at any other suitable times. In a preferred set of variations, S210 is performed at a predetermined frequency (e.g., once per day, more than once per day, less than once per day, etc.) for each participant, but can additionally or alternatively be otherwise performed.

The set of scores determined in S220 preferably includes multiple scores, but can alternatively include a single score. In variations including multiple scores, the scores can be any or all of: independently determined and/or used, determined together and/or aggregated, and/or any combination.

The set of scores are preferably associated with (e.g., attributed to, assigned to, etc.) the participants, wherein the value of the scores can be used, for instance, to determine which participants a coach should reach out to (e.g., first, at all, etc.), how a coach should reach out to the participants (e.g., based on their communication styles, engagement in the program, etc.), and/or any other ways to guide the coaches in reaching out to their participants.

Additionally or alternatively, any or all of the set of scores can be associated with (e.g., attributed to, assigned to, etc.) one or more inputs (e.g., participant messages, coach messages, etc.), one or more outputs, other information on a participant interface, other information on a coach interface, and/or any other suitable information.

The set of scores preferably includes a criticality score (equivalently referred to herein as a critical score and/or urgency score), which is used to determine a critical list and/or grouping of participants (e.g., as described below). The critical score is preferably a binary indicator indicating critical vs. non-critical participants, but can additionally or alternatively be associated with a category, chosen from a continuous spectrum of values, or otherwise determined. The criticality score preferably takes into account clinical information and/or needs of the participant, such as a need to escalate the participant to clinical care and/or clinical coaching. In some variations, for instance, the critical score assesses whether or not and/or to what degree a participant needs escalated care, such as to any or all of: a clinical coach, a clinician (e.g., one of the participant's physicians), an emergency responder (e.g., through calling 911), and/or any other assistance. Additionally or alternatively, the criticality score can take into account non-medical information and/or any other suitable information.

In some variations, the criticality score is determined based partially or fully on health and/or medical information associated with the participants, such as information associated with one or more health conditions (e.g., predetermined, dynamically determined, etc.) of the participant. The inputs used to determine the criticality score can optionally be collected from one or more biometric devices associated with the participant (e.g., glucose meter, blood pressure cuff and/or sensor, heart rate monitor, insulin pump sensor, etc.). Additionally or alternatively, inputs can be collected from any or all of: medical files, other care team members (e.g., clinical coaches), inputs manually entered by the participant (e.g., messages, metrics, etc.), and/or any other suitable input sources. In specific examples, the criticality score is determined based on biometric information associated with the user, such as a glucose level, blood pressure, and/or any other suitable information, wherein the criticality score is increased in an event that the biometric information falls outside of an expected and/or designated range.

The set of scores further preferably includes an impact score, which functions to score the likelihood of a participant to benefit from coach outreach, thereby enabling a prioritization of participants based on a quantification of how much they are predicted to benefit based on the coach reaching out to them.

The impact score is preferably used to determine one or more impact-ranked lists (e.g., as described below), wherein participants with the highest impact scores are at the top of the impact-ranked list. Additionally or alternatively, the impact score or scores can be used in any other suitable ways.

The impact score can include a single score (e.g., overall impact score), multiple scores (e.g., quantifying the predicted impact of different types of coach interactions), and/or any combination of both. The impact score is preferably determined based on any or all of the models described above, further preferably one or more trained models (e.g., optimized for helping a participant in reaching one or more health goals) but can additionally or alternatively be determined by any other models, algorithms, and/or equations. The value of the impact score can be associated with a category, such as high impact, medium impact, and low impact. Additionally or alternatively, the value of the impact score can be determined from a spectrum of possible impact scores (e.g., between 0 and 1, between 0 and 100, etc.), a binary score, and/or any other suitable score.

In preferred variations, the impact score is determined with one or more uplift models (e.g., as described above), wherein the impact score produced by the uplift models reflects the predicted difference in impact (e.g., meaning) between the coach reaching out to the participant (e.g., at that particular moment in time) and the coach not reaching out to that participant, wherein a higher impact score for a participant indicates a higher overall impact/reward for the coach reaching out to that particular participant. Additionally or alternatively, the impact score can be determined based on any other models (e.g., predictive models, regression models, etc.). Further additionally or alternatively, the impact score can optionally be determined based on any other scores, such as any of the other scores described above and/or below (e.g., rapport score).

The uplift model(s) and/or any other models (e.g., as described above) are preferably trained based on historical aggregated inputs (e.g., message behavior, goals, outcomes, etc.) from a set of participants, but can additionally or alternatively be trained based on simulated and/or synthetic data, based only on historical information associated with the particular participant, be untrained, and/or any combination.

The impact score is preferably determined based on current and/or historical information associated with the participant, such as behavior information (e.g., prior interactions with coach, response times, message content and/or length in response to the coach reaching out, progress toward achieving goals, etc.), but can additionally or alternatively be determined based on any or all of the inputs described above.

The set of scores can optionally additionally or alternatively include one or more scores associated with messages of a participant and/or coach, such as to determine a sentiment, emotion, rapport, and/or tone associated with the messages. This score or scores can subsequently be used for instance, to prioritize the coach's interactions with participants showing a high rapport with the coach, as the inventors have discovered that these participants benefit more from coaches reaching out than participants demonstrating a low rapport. This might indicate, for instance, any or all of: that better rapport between participants and coaches correlates with building the participant's trust in the coach and/or program; that participants who get along with and/or are most engaged with their coaches are more willing to engage with the program; that participants who are engaged with messaging depend on and/or expect coach replies to a greater extent than participants who are not as engaged with messaging; that participants who have high rapport with coaches have higher motivation in achieving their goals; and/or this can indicate any other trends or phenomena.

The set of messages scores preferably includes a rapport score, but can additionally or alternatively include scores optimized for other behaviors and/or sentiments associated with the messages.

The message scores preferably take into account of one or more features of a set of one or more messages, such as, but not limited to, any or all of: a content of the message (e.g., topics discussed, words used, tone of words used, etc.); a length of the message (e.g., long vs. short, number of words, etc.); whether proper grammar and/or punctuation is present (e.g., “Okay, thank you so much!”) or not (e.g., “k thanks”); what type of punctuation is used (e.g., a high number of question marks, an exclamation point, a period, none, etc.); whether the message includes follow-up such as a follow-up question to indicate engagement; a tone of the message (e.g., high percentage of positive/optimistic words used, high percentage of negative/pessimistic words used, only neutral words used, etc.); whether or not the writer of the message addresses the recipient (e.g., by name); and/or any other features associated with the content of the message.

In some variations, for instance, a message from a participant stating “ok thanks” is associated with lower rapport (e.g., based on length, punctuation, tone, lack of follow-up, etc.) than a message stating “Thanks, Coach! That's super helpful. Is there anything else I should be doing to make sure I stay on track with my meal prep goals?”.

The message scores can additionally or alternatively take into account one or more temporal parameters associated with messaging of the participant, such as any or all of: a frequency with which the participant messages the coach, an average time since the participant's last message, a response time (e.g., average response time, longest response time, shortest response time, etc.) with which the participant replies to a coach's message, and/or any other parameters. A participant who frequently messages the coach and/or replies quickly to a coach's message can be associated, for instance, with a higher rapport score than a participant who barely engages with the coach.

The message scores can further additionally or alternatively take into account any number of metrics (e.g., progress metrics) associated with the participant's participation in a health program, such as any or all of: a current level and/or status; how quickly the participant performs modules; how quickly the participant reaches milestones; how often the participant logs into the platform; how long per day the participant engages with the platform; and/or any other metrics.

The message score (e.g., rapport score) is preferably determined, at least in part, with natural language processing (NLP) of any or all of the messages written (e.g., typed) by the participant and optionally any or all of the messages written by the coach (e.g., to prompt a message from the participant, in response to a message from the participant, etc.). This can function to determine one or more content features described above and/or any other features.

Additionally or alternatively, the message score can be determined with one or more rule-based models, such as a set of rules (e.g., programmed rules, If/Then rules, comparison with a set of criteria or thresholds, comparison with a lookup table, etc.) with which to determine a message score. In some variations, processing of the message features and/or parameters and/or metrics with a set of rules can be used to determine a level of rapport and/or any other behavior between the participant and the coach.

Further additionally or alternatively, the message score can be determined with one or machine learning models, any or the models described above, and/or any other suitable models, algorithms, equations, and/or tools.

In some variations, one or messages scores (e.g., a rapport score) is factored into and/or used to determine the impact score, such that participants with high rapport scores are generally moved up higher in impact-ranked lists.

In some variations, the impact score takes into account one or more other scores (e.g., rapport score, critical score, etc.) associated with a participant. In specific example, for instance, a participant with a high rapport score increases the impact score. Additionally or alternatively, the scores can be unrelated (e.g., independently determined) or otherwise determined.

The set of scores can optionally additionally or alternatively include a priority score, which functions to quantify a priority associated with the coach reaching out to and/or engaging with the participant. The priority score can be used, for instance, in determining a priority-ranked list (e.g., as described below). Additionally or alternatively, the priority score can be otherwise used.

The priority score is preferably associated with one or more temporal parameters (e.g., time since coach last communicated with a participant), wherein the priority ranked list indicates a grouping of participants to reach out to within a predetermined time period (e.g., end of business day) and/or optionally a temporal order in which to reach out to them. Additionally or alternatively, the priority score (and/or any other scores) can be associated with and/or determined based on a set of rules (e.g., mathematical models) associated with commercial or other business goals of the platform (e.g., communicating with each participant at a predetermined frequency, responding to a participant within a predetermined time frame, etc.). The priority score can be determined independently of other scores (e.g., impact score and/or the critical score), but can additionally or alternatively be determined based on other scores. The priority score can be determined based on the models described above, temporal data (e.g., time since last interaction), and/or any other suitable models, algorithms, or data.

In some variations, for instance, a priority scores is determined for each of the set of participants to ensure that a coach reaches out to the participants within time thresholds predetermined for the program, such as a guarantee to participants that a coach will do any or all of: reach out to a participant at a minimum predetermined frequency (e.g., once per day, once per week, etc.); respond to a message from a participant within a predetermined time (e.g., within at most one day, by close of business, etc.); and/or any other constraints. This can be used, for instance, to provide a coach with a priority-ranked list which a coach can reference to ensure that he or she is meeting minimum program requirements.

The set of scores can additionally or alternatively include any or all of the scores described above and/or any other scores resulting from the models described above.

Additionally or alternatively, any other suitable scores can be determined.

4.3 Method—Assigning a Set of Tags and/or Labels to a Participant S230

The method 200 can include assigning a set of tags and/or labels to one or more participants S230, which functions to provide easy reference (e.g., visual reference) to a coach of one or more features associated with a participant. Additionally or alternatively, the tags and/or labels can function to lend a searchability to a set of participants and/or other information (e.g., individual messages) associated with the participants. In preferred variations, for instance, the coach can search based on label, wherein any participants and/or other information associated with the label is pulled up. In specific examples, any or all of the scores (e.g., impact score, message score such as rapport score, etc.) can be used in organizing these search results. Further additionally or alternatively, the tags and/or labels can function to trigger one or more actions (e.g., escalation to a clinical coach), and/or be used in any other suitable ways.

S230 is preferably performed in response to and/or parallel with S220, but can additionally or alternatively be performed prior to S220, in absence of S220, in response to S210, and/or at any other suitable times. Further additionally or alternatively, S230 can be performed at any or all of: continuously (e.g., at a predetermined frequency), in response to a trigger (e.g., in response to a coach assigning a label, in response to an input being received which triggers the label, etc.), at random intervals, and/or at any other suitable times.

The tags and/or labels can be any or all of: predetermined, dynamically determined, custom (e.g., as determined by a coach and/or coach preference), uniform among multiple coaches and/or care team members (e.g., multiple coaches caring for the same participants, etc.), otherwise determined, and/or any combination. In some variations, the system includes a set of predetermined labels for the coaches to use, which creates a shared taxonomy among the set of coaches, along with the option for the coaches to make their own custom tags (e.g., free text tags).

The tags and/or labels can indicate any or all of: a type of response a coach should consider providing to a participant (e.g., medical vs. health vs. fitness), a time period in which a coach should respond (e.g., to meet a predetermined time limit for responding to a patient, etc.), a type of alert associated with a participant (e.g., change in biometric signal such as blood pressure, change in weight of participant, new message received from participant, etc.), a patient state and/or progress level (e.g., positive progress, no progress, negative progress, healthy, unhealthy, etc.), a level of urgency and/or criticality of a patient state (e.g., highly critical, moderately critical, slightly critical, etc.), one or more features of the participant (e.g., demographic information, location information, etc.), and/or any other suitable indicator(s).

Any or all of the tags and/or labels can be optionally determined, in part or in full, based on the set of scores. Additionally or alternatively, any or all of the labels can be determined independently of the set of scores and/or based on any or all of: the set of inputs (e.g., participant medical information, coach preferences, etc.), a set of outputs, a set of predetermined parameters and/or thresholds (e.g., temporal parameters), and/or any other suitable information.

The tags and/or labels can optionally be determined based on comparing one or more parameters (e.g., from participant inputs, temporal parameters, scores, etc.) with a set of thresholds, but can additionally or alternatively be determined based on any or all of: a set of models (e.g., as described above, additional or alternative to those described above, etc.), a set of algorithms, processing of a set of received inputs, a calendar and/or timer, assigned to a participant (e.g., based on onboarding), a coach input such as a coach selection of a tag and/or label, and/or otherwise determined.

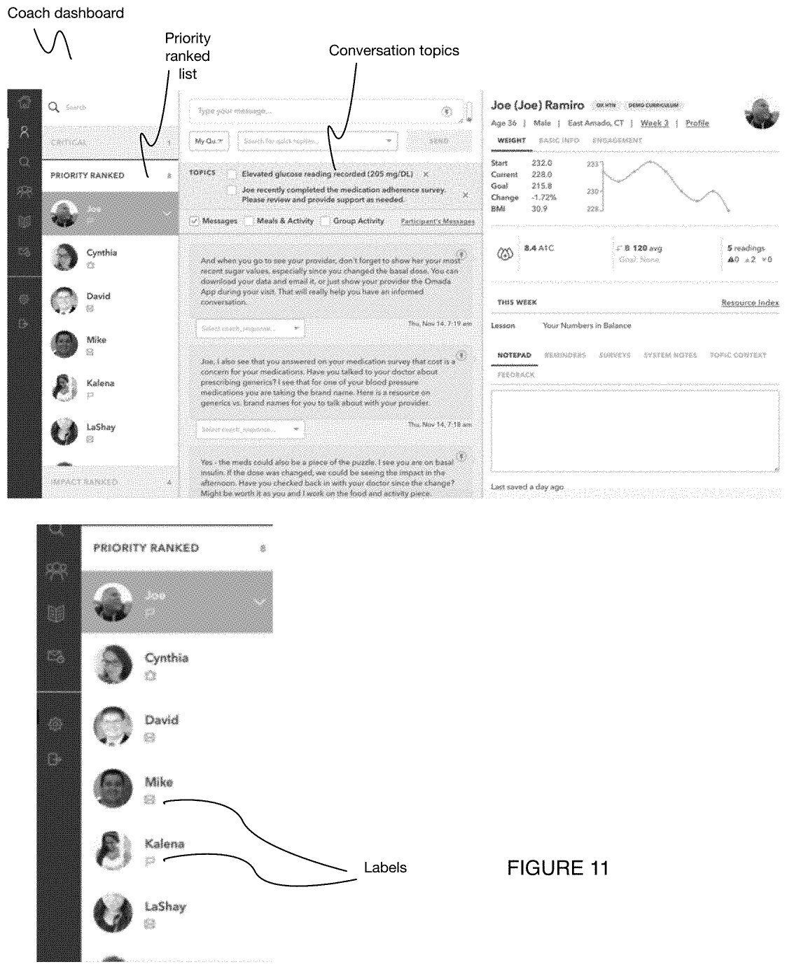

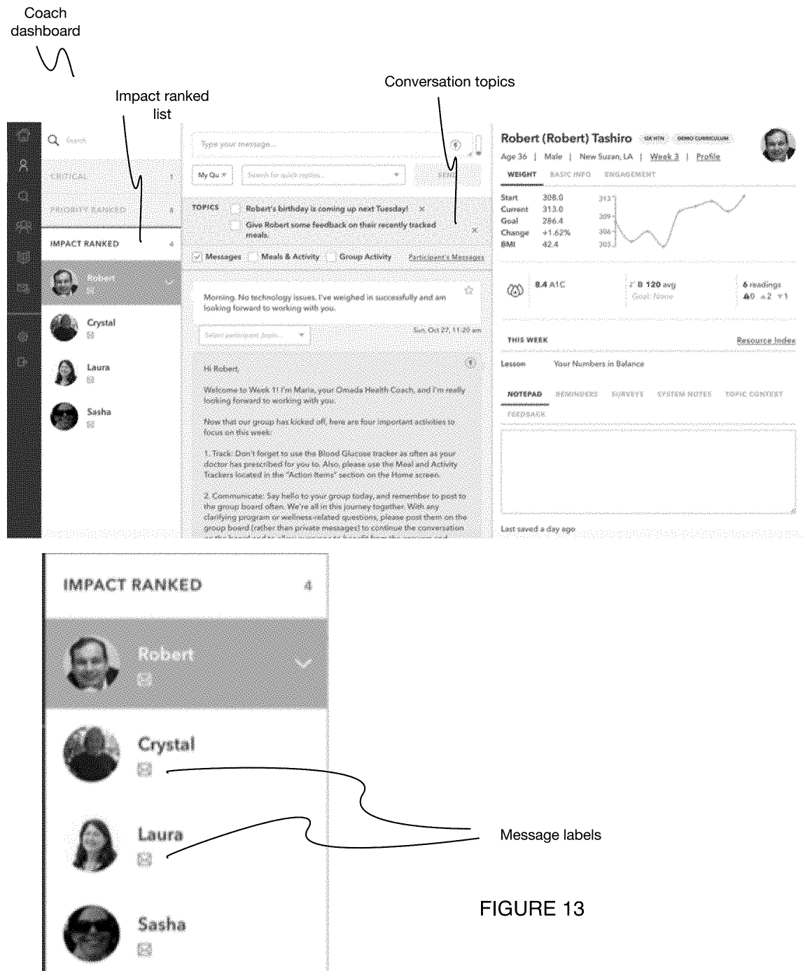

The tags and/or labels are preferably included on a participant identifier (e.g., photo, tab, etc.), such as the participant's tab in a list (e.g., as described below) as shown in FIGS. 11 and 13. Additionally or alternatively, the labels can be indicated on a participant's profile, sent to a coach (e.g., in a message), arranged in a spreadsheet or database, and/or otherwise displayed or communicated (e.g., through an audio notification) to the coach.

The tags and/or labels can indicate any or all of: one or more clinical features and/or conditions of the participant (e.g., high blood pressure, low blood pressure, anxiety, depression, sleep apnea, obstructive sleep apnea, a chronic condition, etc.), which can be any or all of: indicated by the participant (e.g., during onboarding, in a message, in a survey, etc.), indicated based on the participant's medical history, assigned by a clinical coach, and/or otherwise determined. The tags and/or labels can additionally or alternatively indicate whether or not the participant is associated with a pending action item (e.g., action item requested by participant, action item set by coach, action item set by system, etc.), which can function to cue the coach to fulfilling the action item. The tags and/or labels can additionally or alternatively include labels and/or tags indicating which supplementary devices (e.g., blood pressure monitor, glucose meter, scale, insulin pump, mobile user device, desktop user device, etc.) a participant is using, which medications a participant is taking, which features of the program (e.g., meal tracker, fitness tracker, etc.) the participant is taking part in, and/or any other information. The tags and/or labels can additionally or alternatively include labels and/or tags indicating one or more participant features, quirks, and/or behaviors, such as, but not limited to, any or all of: a first language of the participant (e.g., native English speaker, non-native English speaker, etc.); a goal of the participant (e.g., lose weight, manage Type-2 diabetes, etc.); an eating and/or dieting style or history of the participant (e.g., chronic dieter, keto dieter, low carb dieter, low fat dieter, emotional eater, stress eater, etc.); dietary restrictions of the user (e.g., gluten free, vegetarian, vegan, dairy free, etc.); a financial characteristic of the participant (e.g., budget-conscious, non-budget-conscious, etc.); how the coach perceives the participant's engagement (e.g., engaged vs. non-engaged, high rapport vs. low rapport, etc.); an exercise level of the participant (e.g., beginner, intermediate, advanced, etc.); a tech savviness of the participant (e.g., low, moderate, high, etc.); demographic information (e.g., age, gender, sex, race, etc.); triggers for the participant (e.g., sensitivity of stepping on a scale, etc.); a progress level and/or progress trend (e.g., improving, declining, reached a plateau, etc.); an availability of the participant (e.g., on vacation); an occupation and/or type of occupation (e.g., night shift, day shift, frequent traveler, etc.); a relationship status (e.g., single, married, has kids, lives alone, etc.); a motivation level; and/or any other suitable labels and/or tags.

In some variations of the method (e.g., as shown in FIG. 11), users to which any or all of the following labels apply receive tags and/or labels indicating: a flag associated with the participant, such as a flagged medical/biometric parameter (e.g., elevated glucose level, elevated heart rate, etc.), a flagged parameter associated with a health regimen or set of goals of a user (e.g., a weight gain, a weight loss, a drop in exercise, a large amount of consumed calories, a stop in participant recording of meals, etc.), a flagged parameter associated with a communication of the participant (e.g., drop in participant's communication with a coach, an unanswered message sent to the participant, etc.), and/or any other flags.

Additionally or alternatively, the tags and/or labels can include an alarm label (e.g., as shown in FIG. 11), which is determined based on one or more temporal parameters, such as one or more temporal parameters exceeding a threshold. The temporal parameters can indicate any or all of: a threshold time (e.g., 1 day, less than 1 day, greater than 1 day, etc.) has passed since the coach last communicated with the participant, a threshold time has passed since the participant has sent a message to the coach, and/or any other suitable temporal parameters.

Further additionally or alternatively, the tags and/or labels can include a message label (e.g., the letter label in FIGS. 11 and 13), which indicates that the participant has reached out to the coach (e.g., sent a message, made a call, etc.).

Any or all of the tags and/or labels are preferably indicated in a set of ranked lists (e.g., priority-ranked list, impact-ranked list, etc.), but can additionally or alternatively be included in non-ranked lists (e.g., critical list), be used to create a list in response to a coach entering a search term associated with the label and/or selecting the label, be displayed at the user's profile, and/or can be in any other suitable lists and/or features of the dashboard.

In a variation of S230, a set of tags and/or labels are included in the priority-ranked list, which include the label categories of flagged readings (e.g., from one or more sensors, from participant data entries, etc.), alarms, and message indicators, and wherein a set of labels are included in the impact-ranked list, which include message flags.

Additionally or alternatively, a set of tags and/or labels can be otherwise determined and/or implemented.

4.4 Method—Organizing the Set of Participants Based on the Scores and/or the Labels S240

The method 200 includes organizing (e.g., ranking, grouping, batching, etc.) the set of participants based on the scores and/or the labels S240, which functions to efficiently present useful and actionable information to the coaches. Additionally, S240 can function to ensure that a participant in need of coaching (e.g., due to an emergency, due to a high propensity for leaving the program, based on a time since last check-in, etc.) is alerted to the coach. Further additionally or alternatively, S240 can perform any other suitable function(s).

The participants are preferably organized, at least in part, based on the set of scores (e.g., an impact score, a critical score, etc.), but can additionally or alternatively be determined based on a set of labels, and/or any other suitable information (e.g., inputs, outputs, etc.). In some variations, one or more of the set of lists includes only participants associated with a label and/or a score outside of a set of thresholds. Additionally or alternatively, any or all of the lists can include all participants, participants associated with only a subset of labels, and/or any other suitable participants.

S240 can optionally include organizing the participants into a set of lists. Any or all of the lists can be organized such that the order of the participants in the list conveys useful information to a coach, such as any or all of: the potential impact of reaching out to a participant, which participant to reach out to (e.g., first), the urgency of reaching out to a participant, a status of a participant, and/or any other suitable information. Additionally or alternatively, the lists can be absent of an order (e.g., a grouping), or otherwise configured.

In some variations, each participant appears in at most one list, such as at most in one of: a critical list, a priority ranked list, and an impact ranked list. Additionally or alternatively, participants can appear in multiple lists, no lists, or any combination. In specific examples, a participant is placed nearer to the top of the list if the participant appears in multiple lists (e.g., impact and priority).

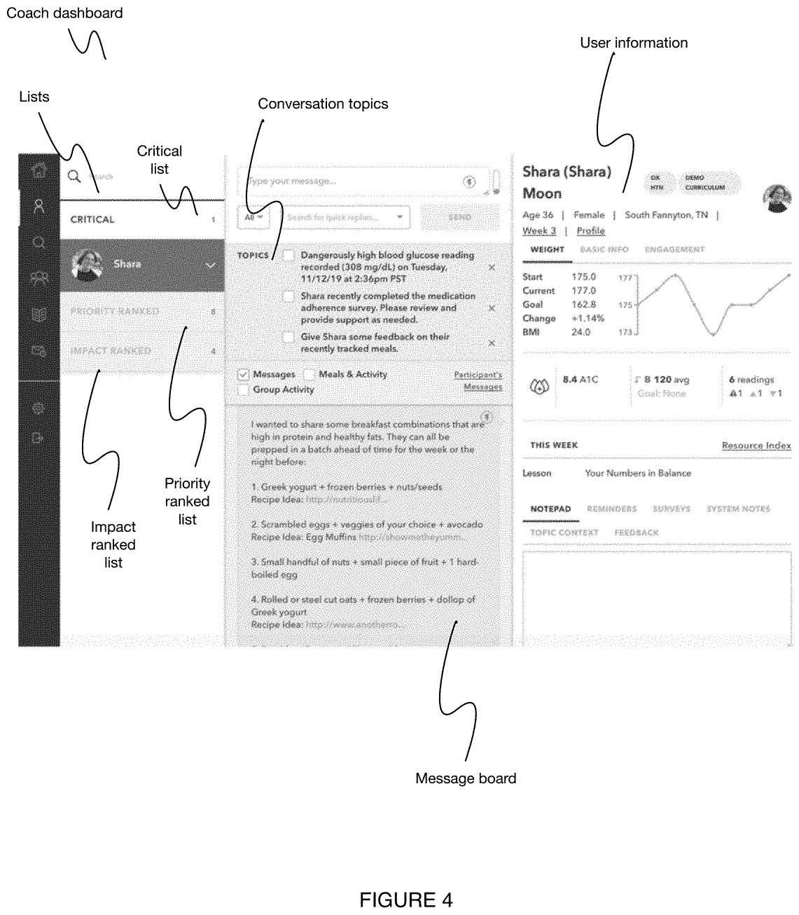

The set of lists can include a critical list (e.g., as shown FIG. 4), which can function to highlight the participants to which the coaches should pay the most immediate attention. The critical list can include participants in need of and/or recommend for any or all of the following from a coach, medical professional, and/or any other suitable individual: an action (e.g., message, notification, advice, etc.); monitoring; viewing of information associated with the participant (e.g., medical information, meal tracker information, fitness activity information, etc.); advice and/or intervention; and/or any other suitable response.

The critical list is preferably associated with participants associated with concerning information, such as an abnormal medical reading (e.g., high glucose level, low glucose level, etc.). Additionally or alternatively, the concerning information can be associated with any other information (e.g., lack of recent communication from the participant). In some variations, the critical list includes participants having a flagged reading label.

Additionally or alternatively, the critical list and/or any other lists (e.g., priority-ranked, impact-ranked, etc.) can be associated with any number of temporal parameters, such as a recommended and/or enforced time for a coach or other individual (e.g., medical professional) to respond to participants in the critical list (e.g., on an individual critical participant basis, for all critical participants, etc.). The time to respond can be predetermined, dynamically determined (e.g., based on the overall information associated with a participant, based on aggregated information for all participants, based on the number of participants in the critical list, etc.), determined based on how long the participant has been part of the platform (e.g., based on communication thresholds which change based on time in program), and/or determined in any other suitable ways. The temporal parameters (e.g., time limits, suggested time to completion, etc.) can optionally be implemented through any or all of: a calendar/clock (e.g., a timer initiated in response to a coach communication to the participant), a notification and/or reminder to the coach (e.g., to alert him or her of the time remaining to respond), an indication on the participant profile, and/or any other suitable indicator. Additionally or alternatively, the temporal parameter can be indirectly indicated to the coach based on a suggested temporal parameter associated with all participants in the critical list, such as a conventional understanding that coaches should respond to critical participants within an established time frame (e.g., before the end of the working day). Further additionally or alternatively, any or all of the lists can include any number of temporal parameters, suggested and/or enforced in any suitable way(s).

The set of lists can additionally or alternatively include a set of one or more ranked lists, which can optionally function to guide a coach in the subset of participants he or she should reach out to (e.g., based on one or more predetermined time periods) and optionally the order in which the coach should reach out to the subset of participants. The subset and/or order can be determined based on any or all of: the urgency of responding to a participant (e.g., based on the time since the coach has last reached out to the participant), a score associated with the participant, a label associated with a the participant, historical information associated with the participant, and/or any other suitable information.

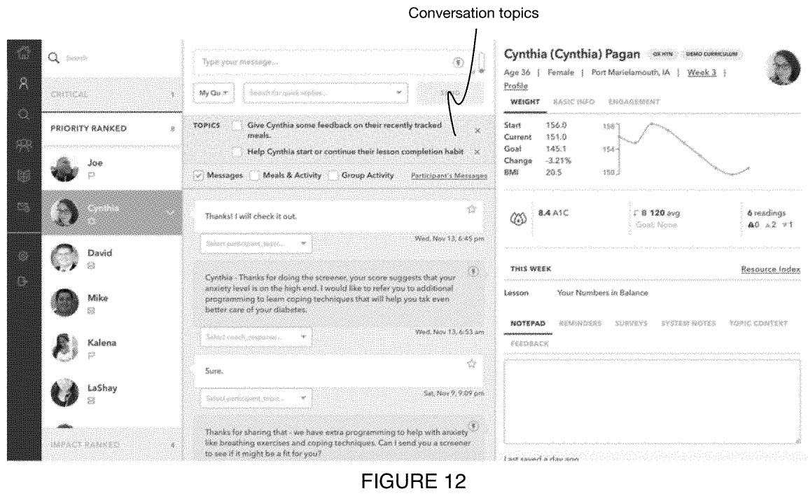

The set of ranked lists can optionally include a priority ranked list (e.g., as shown in FIG. 11, as shown in FIG. 12, etc.), which functions to list the participants (e.g., the subset of participants) associated with a label. The priority ranked list preferably includes a group of participants to reach out to within a predetermined time period (e.g., end of business day), but can additionally or alternatively be ordered or otherwise un-ordered.

The set of lists can optionally include an impact-ranked list (e.g., as shown in FIG. 13), which functions to communicate to the coach which participants have the highest likelihood of positive change after interacting with the coach. The impact-ranked list is preferably determined based on impact scores determined from a set of models, further preferably a set of uplift models as described above, but can additionally or alternatively be determined based on any other inputs, models, lists, scores, labels, and/or other information.

In preferred variations, the method can include producing any number of impact-ranked lists based on any or all of the tags and/or labels as described above (and/or any other suitable tags and/or labels), and/or based on any other information (e.g., from a keyword search by the coach, from a search of message topics and/or content, etc.), wherein the results (e.g., participants) associated with the tag/label and/or search term are arranged in a ranked list based on their impact score (e.g., with participants having the highest impact score at the top of the list). In specific examples, for instance, a coach can search for all participants living in a food desert, wherein the participants associated with this label are organized based on their impact scores. Additionally or alternatively, the participants can be organized based on any other scores (e.g., rapport score) and/or otherwise organized.

In variations including multiple lists, the lists can be determined independently, partially or fully determined based on each other (e.g., priority ranked list takes into account grouping of critical participants), and/or any combination of both. The lists can optionally be associated with an explicit or implied order to handle the participants in the list (e.g., critical list followed by priority-ranked, followed by impact-ranked), but can alternatively be unordered.

S240 can additionally or alternatively include batching any or all of a coach's participants, which can function to increase an efficiency of a coach in working through a set of participants to address. Batching can occur within a set of lists, outside of a set of lists, independently of a set of lists, and/or in any other suitable way. The batching can be facilitated through a set of labels (e.g., as described above, different than those described above, etc.) associated with the participant, but can additionally or alternatively be determined based on any other suitable information (e.g., set of inputs, scores, place in a list, etc.). In some variations, for instance, rather than working top to bottom through an impact ranked list, coaches can work in participant batches identified through a set of models, wherein in specific examples, the batches include participants predicted to experience churn (e.g., from meal tracking, from lesson participation, etc.), participants showing signs of emotional distress (e.g., based on the language they're using in conversations), and/or any other suitable grouping of participants.

In preferred variations, S240 includes organizing the set of participants into a set of lists including a critical list, a priority-ranked list, and one or more impact-ranked lists, wherein the participants can optionally be further batched (e.g., based on tags and/or labels), which collectively functions to assist a coach in efficiently interacting with his or her participants.

In a first set of examples, S240 includes creating a set of impact-ranked lists based one or more tags and/or labels, wherein the coach can search by tag/label.

4.5 Method—Determining a Set of Conversation Topics Associated with a First Participant in the Set of Participants S250

The method 200 can optionally include determining a set of conversation topics associated with a participant in the set of participants based on the set of scores and/or labels S250, which functions to determine and suggest a set of conversation topics to a coach for a particular participant, wherein the set of conversation topics are predicted to be most optimal (e.g., most impactful, most relevant, most effective, etc.) for each participant in achieving his or her health goals, thereby maximizing an efficiency of each coach. This can in turn can function to: improve an efficiency of the coach (e.g., by saving time coach would have to spend manually coming up conversation topics); help a coach better engage with a participant (e.g., by targeting the most useful conversations to have); and/or perform any other suitable functions.

Determining (e.g., selecting, choosing, creating, identifying, matching, etc.) a set of conversation topics can include any or all of: selecting one or more conversation topics from a library or database (e.g., a lookup table), determining one or more conversation topics based on a decision tree, selecting and/or generating (e.g., generating any or all of the text of a conversation topic) one or more conversation topics based on a set of models (e.g., deep learning models, models described above, different models than those described above, linguistics models, etc.), predicting one or more conversation topics (e.g., predicting most relevant conversation topics for a particular user, predicting most effective/impactful conversation topics for a particular user, predicting a conversation topic from a predetermined set, generating a predicted optimal conversation topic, etc.), and/or otherwise determining a set of conversation topics.

The set of conversation topics are preferably determined based on participant information, such as any or all of the participant information (e.g., sensor information, historical information, progress information, etc.) included in the participant profile. The conversation topics can additionally or alternatively take into account any or all of: participant scores, participant labels, participant ranking in a list, and/or any other suitable information. The conversation topics are further preferably determined based on information aggregated from a set of multiple participants, such as based on a decision tree determined based on and optionally updated with aggregated participant information. Additionally or alternatively, the set of conversation topics can be determined based on information associated with a single participant (e.g., historical information associated with the participant), and/or any other information.

Any or all of the conversation topics can be determined/selected based on a likelihood of inducing a positive behavior change in a participant. In some variations, for instance, any or all of a decision tree (and/or any other suitable models, algorithms, etc. for determining the conversation topics) can be determined based on aggregated, historical information which associates conversations with behavior change (e.g., indirectly, directly, though regression modeling, etc.). The behavior changes are preferably associated with (e.g., shown to lead to) one or more outcome key drivers, such as any or all of: weight loss of a participant, medical improvement metric (e.g., glucose level) of a participant, fitness improvement metric (e.g., amount of activity performed each day), and/or any other suitable metrics. The conversation topics can additionally or alternatively be determined based on any other suitable information.

Determining the set of conversation topics preferably includes predicting the most relevant and effective topics for a participant or group of participants (e.g., batched participants). This can be based on any or all of the inputs described above, such as any or all of: the participant's goals, progress, interactions with his or her coach, communication style, historical information (e.g., previous behavior changes), and any other participant information. Additionally or alternatively, information associated with aggregated participants (e.g., historical information indicating which conversation topics led to positive behavior change) can be used, information associated with the coach can be used, and/or any other suitable information can be used.

In some variations, determining the set of conversation topics includes comparing the set of conversation topics with previous conversation topics and preventing and/or eliminating conversation topics which have been previously implemented (e.g., to reduce repeating information). Additionally or alternatively, the conversation topics can be repeated (e.g., to reinforce a topic, based on a predicted behavior change, etc.) and/or otherwise suggested.

S250 can additionally include automatically determining the messages themselves associated with the conversation topics, wherein the messages can be auto-populated, auto-filled, suggested, and/or otherwise provided to a coach and/or implemented.

S250 can additionally or alternatively include preventing a coach for repeating exact and/or nearly exacting information to a participant, wherein repeating the same message can be associated with diminished trust, as it can cause the participant to feel that the coach is not paying attention to him or her specifically. In some variations, for instance, because a message from the coach is sent to the participant, the text of the comparison is compared with previous messages sent to the participant (e.g., by the same coach, by a different coach, etc.), such as through string matching, wherein if the message exactly or substantially (e.g., includes the same content but different working) matches a prior message, it is flagged to the coach and/or not sent. This can optionally first involve distinguishing substantive messages (e.g., those involving recommendations, those providing particular content, etc.), from non-substantive messages (e.g., greetings, sign-off messages, etc.), such that only substantive messages are compared, but can additionally or alternatively be otherwise performed.

S250 can optionally include identifying and/or organizing any or all of the participants based on a set of conversation topics. In some variations, for instance, additional or alternative to those described above, coaches can identify which participants to reach out to based on the recommended conversation topics for the participant. This can include for instance, any or all of: batching participants together based on their associated recommendation topics so they can be replied to contemporaneously and/or together (e.g., with the same message, with similar messages, with different messages, with auto-filled messages, with auto-generated messages, etc.); determining a priority (and/or impact, criticality, urgency, etc.) of reaching out to a particular participant based on the content of the recommended conversation topics (e.g., short-term guidance vs. long-term guidance, medical advice vs. general conversation, etc.); and/or assisting a coach in any other suitable way(s). Additionally or alternatively, conversation topics can be determined and/or applied based on the set of labels and/or scores.

S250 is preferably performed in response to S240, but can additionally or alternatively be performed prior to S240, wherein any or all of the rankings are determined based on the conversation topics (e.g., content, category, number, etc.). Can further optionally be performed prior to S230 and/or S220, wherein any or all of the labels and/or scores are determined based on the conversation topics (e.g., content, category, number, etc.).

4.6 Method—Recommending the Set of One or More Conversation Topics to a Coach Associated with the Participant at a Dashboard S260

The method 200 can optionally include recommending the set of one or more conversation topics to a coach associated with the participant at a dashboard S260, which functions to enable the coach to easily initiate a conversation with a user.

The conversation topics can optionally be ordered and/or grouped, which can indicate any or all of: the order in which a set of topics should be initiated with a participant, a ranking of the likelihood that a topic will induce a positive behavior change, an ordering of topics based on when they were last received by the participant, and/or any other suitable order. Alternatively, the topics can be unordered.

Examples of conversation topics can be seen in FIGS. 4 and 11-13.

S260 can further include receiving a selection of a conversation topic from a coach. This can subsequently trigger any or all of: auto-generating a message for a coach, auto-filling any or all of a message initiated by a coach, and/or any other actions configured to assist a coach in quickly, accurately, and/or otherwise optimally writing a message. In these variations, auto-generating and/or auto-filling messages can include any or all of: taking into account a writing style of the coach to maintain his or her personal touch, taking into account previous messages of the coach to the participant (e.g., to prevent redundancy, to auto-fill, etc.), identifying a subset or full set of participants that the message applies to (e.g., which can subsequently trigger the message getting sent to all of these participants through a central broadcast feature), and/or taking into account any other suitable information. This can be achieved through any or all of: a set of predictive/deep learning models (e.g., trained on previous messages of the coach, trained on messages from an aggregated set of participants, trained on simulated messages, etc.) and/or any other suitable models, decision trees, and/or other tools. In some variations, the method can include enabling a coach to label and store a set of commonly-used responses (e.g., favorite recipes, workout plans, commonly-used responses, etc.), such that the coach can initiate and have the associated text auto-filled upon initiating the text (e.g., through the label, through a keyboard shortcut, etc.).

### 4.7 Method—Receiving an Input S270

The method 200 can include receiving an input S270 from any or all of: the coach (e.g., a message to a participant based on a conversation topic), a participant (e.g., a message from the participant), a component of the system and/or an external source (e.g., sensor data indicating a behavior change, a progress update associated with a participant, a client application, etc.), and/or any other suitable information source. The inputs can be any or all of those described previously and/or different than those described previously.

### 4.8 Method—Updating the Dashboard Based on the Input S280

The method 200 can include updating a dashboard (e.g., coach dashboard, participant dashboard, etc.) based on the inputs S280, which functions to dynamically adjust a dashboard to include up-to-date information.

The features associated with one or more participants can optionally be updated such as any or all of: a score, label, set recommended conversation topics (e.g., to eliminate a conversation topic just used), place in a ranking, and/or any other suitable features.

The features associated with an aggregated set of participants (e.g., any or all of the participants associated with the coach, participants associated with multiple coaches, all participants, etc.) can optionally be updated, such as through updates to any or all of: overall rankings, eliminating a ranking (e.g., when critically ranked list includes no users), adding a ranking, updating an aggregated set of conversation topics (e.g., based on the effectiveness of a conversation topic), and/or any other suitable features.

Additionally or alternatively, any other features of a dashboard can be adjusted.

### 4.9 Method—Triggering an Action S290

The method 200 can optionally include triggering an action S290, which functions to utilize any or all of the processes described above and/or their associated information to better coach the participants. Additionally or alternatively, S290 can function to trigger an action which improves an efficiency for one or more coaches; updates one or more models based on new information; and/or can perform any other suitable functions.

S290 can be performed in response to any or all of the processes described above, such as in response to any or all of: S210, S220, S230, S240, S250, S260, S270, S280, and/or at any other suitable times. S290 can additionally or alternatively be performed in absence of any of the processes described above; in response to a trigger (e.g., based on a score, based on a label, based on a message, etc.); continuously (e.g., at a predetermined frequency); at random intervals; and/or at any other suitable times.

S290 can optionally include updating one or more models (e.g., as described above), such as based on an input received in S270 and/or based on any other processes and/or information in the method, which functions to dynamically adapt the set of models (and/or algorithms, decision trees, etc.) associated with the method 200 based on new information, which can subsequently function to make the models more robust.

S290 can optionally include tracking one or more outputs, which preferably include a behavior change (e.g., increase in weight loss, increase in exercise, decrease in severity of a medical problem, etc.) and/or a set of outcome key drivers, such as weight loss, and correlating (e.g., through regression modeling) the interactions with the coach to the outcomes. If a particular conversation topic and/or other interaction with a participant leads to a positive behavior change, for instance, a model can be updated to reinforce the conversation topic/action that led to this, such that the same response might be tried with a similar participant. If a particular conversation topic and/or other interaction with a participant leads to a negative behavior change, the model can be updated to eliminate or downgrade (e.g., decrease a weighting factor of) the particular action or response, such that a different response might be tried with a similar participant. This information can then be used for any or all of: retraining one or more models, updating participant information (e.g., scores, labels, etc.), and/or can be otherwise used.

S290 can optionally include triggering one or more actions, such as any or all of: escalating a participant (e.g., to a clinical coach, to a different coach, to emergency services, to a help hotline, etc.); reassigning a participant (e.g., to another coach, to another care team member, etc.); automatically assigning or reassigning one or more labels (e.g., to engage more, to engage less, etc.); reorganizing one or more lists (e.g., moving participant to bottom once coach has messaged participant); removing the participant from the program (e.g., in response to an inappropriate message); establishing communication (e.g., messaging, phone call, etc.) between coaches and/or other care team members; establishing communication between participants (e.g., to facilitate a chat group among participants associated with the same labels); and/or any other actions. In some variations, for instance, tagging a participant and/or a message with an indication that the participant has diabetes can trigger an escalation of the participant to a clinical coach.

Additionally or alternatively, S290 can include any other suitable processes.

### 4.95 Method—Repeating any or all of the Above Processes

The method 200 can include repeating any or all of the above processes, which can function to apply the method 200 to multiple participants and/or multiple coaches. Additionally or alternatively, the method 200 can function to generate training data with which to train and/or refine one or more models of the system and/or perform any other suitable function(s).

### 5. Variations

In a variation of the method 200, the method is configured for at least: reducing churn of participants and therefore detecting a sign that a participant may disengage with the platform and triggering the coach to interact with the participant; recommending participant-specific conversation topics for a coach; auto-generating and/or auto-filling messages for coaches; and prioritizing which participants a coach should reach out to, and includes, for each coach: collecting participant information at a remote computing system including at least participant demographic information, meal and fitness inputs, and sensor information (e.g., weight, pedometer inputs, etc.); providing any or all of the participant information at the participant profile on the coach's dashboard; processing the participant information for each of the set of participants with a set of models implementing uplift modeling, wherein the set of models determine at least a set of impact scores identifying which participants are most likely to be positively impacted (e.g., as reflected in a behavior change) by an interaction with a coach; determining a set of labels associated with a subset of participants based on any or all of the scores, processing, and participant information; determining a set of lists based on the processing and the participant information (and optionally the set of scores and/or set of labels), wherein the set of lists include a critical list, a priority-ranked list, and an impact-ranked list; recommending a set of communication topics for each participant to the coach based on a set of decision trees determined based on aggregated participant information from multiple coaches; optionally auto-generating and/or auto-filling a message for the coach based on his or her selection of a conversation topic; receiving an input associated with the patient (e.g., a behavior change); and updating the models and/or decision trees based on the input.

In a second variation of the method 200, the method includes any or all of: collecting participant information at a remote computing system such as participant demographic information, meal and fitness inputs, and/or sensor information (e.g., weight, pedometer inputs, etc.); providing any or all of the participant information at the participant profile on the coach's dashboard; processing the participant information for each of the set of participants with a set of models (e.g., uplift models, programmed rules, etc.), wherein the set of models determines at least a set of impact scores identifying which participants are most likely to be positively impacted (e.g., as reflected in a behavior change) by an interaction with a coach and optionally any other scores (e.g., priority score, critical score, rapport score); determining a set of labels associated with a subset of participants based on any or all of the scores, processing, and participant information; determining a set of lists based on the processing and the participant information (and optionally the set of scores and/or set of labels), wherein the set of lists include a critical list, a priority-ranked list, and an impact-ranked list; optionally recommending a set of communication topics for each participant to the coach based on a set of decision trees determined based on aggregated participant information from multiple coaches; optionally auto-generating and/or auto-filling a message for the coach based on his or her selection of a conversation topic; receiving an input associated with the patient (e.g., a behavior change); optionally updating the models and/or decision trees based on the input; optionally triggering another action; and/or any other suitable processes.

Additionally or alternatively, the method 200 can include any other suitable processes.

Although omitted for conciseness, the preferred embodiments include every combination and permutation of the various system components and the various method processes, wherein the method processes can be performed in any suitable order, sequentially or concurrently.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A method for automatically improving an efficiency of coaching a set of multiple participants in a digital health program, the method comprising:
receiving, at a coach interface associated with a coach, a set of inputs associated with the set of multiple participants, wherein the set of multiple participants is assigned to the coach;
with a set of models, wherein the set of models comprises a trained uplift model, automatically determining a set of scores for the set of multiple participants, wherein the set of scores comprises:
a criticality score;
a priority score; and
an impact score;
receiving a set of label assignments associated with the set of multiple participants;
automatically organizing the set of multiple participants into a set of multiple lists at the coach interface, wherein automatically organizing the set of multiple participants comprises:
organizing the set of multiple participants into a first list based on the set of criticality scores; and
organizing the set of multiple participants into a second list based on the set of priority scores; and
in response to receiving a search term from the coach, organizing the set of multiple participants into a third list based on the search term, the set of labels, and the set of impact scores;
receiving an input from the coach at the coach interface based on the set of lists; and
automatically triggering an action based on the input.

2. The method of claim 1, wherein triggering the action comprises automatically reorganizing the set of lists based on the input.

3. The method of claim 2, further comprising triggering a second action based on a new label assignment in the set of label assignments, wherein the second action comprises an assignment of a second coach to the participant, wherein the second coach is a clinician.

4. The method of claim 1, wherein the set of inputs comprises, for each of the set of multiple participants, a set of messages exchanged between the participant and the coach, and wherein the method further comprises, for each of the set of multiple participants, determining a rapport score based on the set of messages exchanged between the participant and the coach.

5. The method of claim 4, wherein each rapport score is further determined based on a set of features associated with the set of messages, wherein the set of features comprises at least a length and a tone of the set of messages.

6. The method of claim 5, wherein the set of impact scores is further determined based on the set of rapport scores associated with the set of participants

7. The method of claim 1, wherein the priority score is determined based on a set of temporal parameters associated with the digital health program

8. The method of claim 7, wherein the set of temporal parameters comprises a predetermined maximum threshold amount of time relative to the time a last message of the participant was sent to the coach.

9. The method of claim 1, wherein the set of labels comprises a set of health condition labels, wherein the set of health condition labels is associated with only a subset of the set of multiple participants, and wherein the criticality score is determined based on the set of health condition labels.

10. The method of claim 9, wherein the criticality score is a binary score.

11. The method of claim 1, wherein the input from the coach comprises a message to a first participant of the set of multiple participants, and wherein the method further comprises, in response to receiving the message, comparing the message with a prior set of messages sent from the coach to the first participant, wherein in an event that the message matches a prior message, the method further comprises blocking the message from being sent.

12. The method of claim 1, wherein the impact score is determined based on the uplift model, and wherein the criticality and priority scores are determined with a subset of the set of models, wherein the subset does not include the uplift model.

13. A system for organizing a coach interface for coaching a set of multiple participants assigned to the coach in a digital health program, the system comprising:
a set of participant interfaces associated with the set of multiple participants, wherein the set of participant interfaces receives a set of participant inputs from the set of participants, and wherein the set of participant interfaces comprises a first messaging platform;
the coach interface, wherein the coach interface comprises a second messaging platform in communication with the first messaging platform;
a remote computing system in communication with the set of participant interfaces and the coach interface, wherein the remote computing system comprises a set of models, wherein the set of models comprises an uplift model, and wherein the remote computing system:
receives the set of participant inputs;
automatically determines, with the set of models, a set of scores for the set of multiple participants, wherein the set of scores comprises:
a criticality score;
a priority score; and
an impact score;
receives a set of label assignments associated with the set of multiple participants;
automatically organizes the set of multiple participants into a set of multiple lists at the coach interface, wherein automatically organizing the set of multiple participants comprises:
organizing the set of multiple participants into a first list based on the set of criticality scores; and
organizing the set of multiple participants into a second list based on the set of priority scores; and
in response to receiving a search term from the coach, organizing the set of multiple participants into a third list based on the search term, the set of labels, and the set of impact scores;
receives an input from the coach at the coach interface based on the set of lists; and
automatically triggers an action based on the input.

14. The system of claim 13, wherein the action comprises a reorganization of the set of lists based on the input.

15. The system of claim 13, further comprising a clinical coach interface in communication with the remote computing system, wherein the remote computing system is further configured to establish communication between the clinical coach interface and a first participant interface of the set of participant interfaces in response to a label assignment of the set of label assignments associated with a first participant.

16. The system of claim 13, wherein the set of inputs comprises, for each of the set of multiple participants, a set of messages exchanged between the participant and the coach, and wherein the remote computing system further determines, for each of the set of multiple participants, a rapport score based on the set of messages exchanged between the participant and the coach.

17. The system of claim 16, wherein the rapport score is further determined based on a set of features associated with the set of messages, wherein the set of features comprises at least a length and a tone of the set of messages

18. The system of claim 17, wherein the set of impact scores of the set of multiple participants is further determined based on the set of rapport scores associated with the set of multiple participants.

19. The system of claim 13, wherein each priority score is determined based on a set of temporal parameters associated with the digital health program.

20. The system of claim 19, wherein the set of temporal parameters comprises a predetermined maximum threshold amount of time relative to the time a last message of the participant was sent to the coach.
